(19) United States
(12) Patent Application Publication
(10) Pub. No.: US 2022/0280472 A1
(43) Pub. Date: Sep. 8, 2022

(54) TREATMENT OF PULMONARY FIBROSIS USING INHIBITORS OF NEU1 SIALIDASE

(71) Applicants: University of Maryland, Baltimore, Baltimore, MD (US); The Governors of the University of Alberta, Edmonton (CA); The United States Government as represented by the Department of Veterans Affairs, Washington, DC (US)

(72) Inventors: Simeon E. Goldblum, Baltimore, MD (US); Sergei P. Atamas, Ellicott City, MD (US); Irina G. Luzina, Ellicott City, MD (US); Christopher W. Cairo, Edmonton (CA)

(21) Appl. No.: 17/625,059

(22) PCT Filed: Jul. 8, 2020
(86) PCT No.: PCT/US2020/041212

§ 371 Date: Jan. 5, 2022

Related U.S. Application Data
(60) Provisional application No. 62/871,497, filed on Jul. 8, 2019.

(51) Int. Cl. A61K 31/351; A61K 31/713; A61P 11/00

(52) U.S. Cl. CPC A61K 31/351; A61K 31/713; A61P 11/00

(57) ABSTRACT

The present invention provides a method for treating a fibrotic lung disease or fibrotic lung condition in a subject that involves an increase in NEU1 expression and/or activity, comprising administering to the subject an effective amount of an agent that inhibits the activity of NEU 1 sialidase, thereby treating the fibrotic lung disease or fibrotic lung condition in the subject.

### CROSS REFERENCE TO RELATED APPLICATIONS

This application claims the benefit of U.S. Provisional Appl. No. 62/871,497, filed Jul. 8, 2019, the contents of which are hereby incorporated by reference in their entirety.

### STATEMENT OF FEDERALLY SPONSORED RESEARCH AND DEVELOPMENT

This invention was made with government support under VA Merit Award Number I01BX002352 awarded by United States Department of Veterans Affairs. The government has certain rights in the invention.

### INCORPORATION-BY-REFERENCE OF MATERIAL SUBMITTED ELECTRONICALLY

Incorporated by reference in its entirety herein is a computer-readable sequence listing submitted concurrently herewith and identified as follows: One 5,720 Byte ASCII (Text) file named “Sequence_listing_ST25.txt,” created on Jul. 8, 2020.

### FIELD OF THE INVENTION

The field of the invention relates generally to therapeutics and medicine. More specifically, the field of the invention relates to therapies for pulmonary fibrosis.

### BACKGROUND

Pulmonary fibrosis is a tissue response to multiple, diverse insults to the lung and can complicate specific disease states. The fibrotic process can be relentless and irreversible, as is the case with Idiopathic Pulmonary Fibrosis (IPF), replacing functional lung parenchyma with scar tissue, distorting lung architecture, and compromising lung function. Patients with IPF survive only 2-3 years after diagnosis, making it the most severe form of pulmonary fibrosis. Although numerous host cells, their surface receptors, ligands, and downstream signaling elements, as well as extracellular matrix molecules all have been implicated in IPF pathogenesis, no unified mechanistic model or effective therapeutic intervention has been established.

Prior to the cloning and identification of the four-known human sialidases, increased sialidase activity was reported in bronchoalveolar lavage fluid (BALF) collected from IPF patients. These four sialidase/neuraminidase (NEU)s have been identified as NEU1, -2, -3, and -4. NEU1 is localized to lysosomes where it resides in a multienzyme complex, with protective protein/cathepsin A (PPCA) and β-galactosidase. NEU2 is found in the cytosol, NEU3 is associated with the plasma membrane, and NEU4 is located in mitochondria. As members of the NEU superfamily, each NEU contains one or more of the conserved Asp box, and an amino acid sequence composed of SXDXGXTW, together with the (F/Y) RIP motif, composed of XPRP, where x represents variable residues. Although the overall sequence identities between the four NEUs are low, their catalytic domains share a common six-bladed β-propeller fold architecture. Each NEU selectively hydrolyzes specific glycosidic linkages between sialic acid residues and the subterminal sugar of glycoconjugates.

Currently there is no effective therapy for IPF and other fibrotic lung diseases and conditions. These conditions are often fatal. Accordingly, what is needed are effective treatments for IPF and other fibrotic lung diseases and conditions, such as those associated with connective tissue disorders and those caused by exposure to radiation and/or noxious chemical compounds.

### SUMMARY

We previously established NEU1 as the predominant sialidase in multiple human lung cell types that participate in IPF pathophysiology, including airway epithelia, microvascular endothelia, and fibroblasts. More recently, we demonstrated elevated expression of NEU1 sialidase in the lung tissues of IPF patients. NEU1 immunostaining was consistently more intense in the lung tissue of IPF patients compared to that seen in healthy tissues. The NEU1 immunostaining was most pronounced in the bronchial epithelium, including the brush border, and the vascular endothelium. NEU1-positive fibroblasts were seen within areas of fibrosis and NEU1 expression in lung fibroblasts harvested from IPF patients was elevated at both the mRNA and protein levels compared to that detected in healthy donor cells. In another study, NEU1 immunostaining was increased in the lungs of only one of three IPF patients. Moreover, in these same studies, increased immunostaining of NEU2, -3, and -4 was found as well. These combined data indicate that expression of one or more sialidases is elevated in the lung tissues of IPF patients.

Provided herein are methods for treating fibrotic lung diseases and conditions that involve an increase in NEU1 expression and/or activity, by administering to a patient in need thereof an effective amount of an agent that inhibits a NEU1 sialidase. The NEU1 inhibitor can be a NEU1-selective inhibitor. Examples of such NEU1-selective inhibitors are described herein.

In some embodiments, the fibrotic lung disease or condition is idiopathic pulmonary fibrosis.

In some embodiments, the fibrotic lung disease or condition is associated with a connective tissue disorder.

In some embodiments, the fibrotic lung disease or condition is selected from the group consisting of sarcoidosis, allergic pneumonia, pneumoconiosis, drug-induced fibrosis, radiation-induced fibrosis, noxious chemical compound-induced fibrosis, and fibrogenic alveolitis associated with collagen vascular disease.

In some embodiments, the agent reduces or prevents myofibroblast accumulation in the subject.

In some embodiments, the agent reduces dyspnea caused by the fibrotic lung disease or condition in the subject.

In some embodiments, the NEU1-selective inhibitor is compound C9-BA-DANA.

In some embodiments, the NEU1-selective inhibitor is compound III-32B5.

In some embodiments, the agent is an antibody.

In some embodiments, the agent comprises a nucleic acid molecule comprising a sequence that binds to at least a portion of a nucleotide sequence of NEU1.

In some embodiments, the nucleotide sequence of NEU1 is SEQ ID NO: 1.

In some embodiments, a portion of the nucleic acid molecule is at least 70%, 75%, 80%, 85%, 90%, 91%, 92%, 93%, 94%, 95%, 96% 97%, 97%, 98% or 99% complementary to at least a portion of SEQ ID NO:1.

In some embodiments, the agent comprises a DNA molecule or an RNA molecule.

In some embodiments, the agent comprises an anti-sense DNA molecule or an anti-sense RNA molecule.

In some embodiments, the agent comprises a small interfering RNA (siRNA) molecule.

In some embodiments, the agent comprises a small hairpin RNA (shRNA) molecule.

In some embodiments, the agent comprises a nanoparticle comprising a nucleic acid.

In some embodiments, the agent comprises an expression vector.

In some embodiments, the expression vector is a viral vector or a non-viral vector.

In some embodiments, the viral vector is an adenoviral vector, an adeno-associated viral vector, a lentiviral vector, or a retroviral vector.

In some embodiments, the expression vector is a lentiviral vector.

In another aspect, the invention provides a composition for treating a fibrotic lung disease or condition in a subject, the composition comprising an effective amount of an agent that inhibits the activity of NEU1 sialidase and a pharmaceutically acceptable carrier.

In some embodiments, the composition comprises a nucleic acid molecule that comprises a nucleotide sequence that binds to at least a portion of a nucleotide sequence of NEU1.

It is to be understood that both the foregoing general description and the following detailed description are exemplary, and thus do not restrict the scope of the invention. Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The skilled artisan will understand that the drawings, described below, are for illustration purposes only. The drawings are not intended to limit the scope of the present teachings in any way.

FIG. 1. Broad-spectrum Sialidase Inhibition Protects Against Lymphocyte Infiltration and Collagen Deposition in the Lungs of Bleomycin-Challenged Mice. (A) Wild-type female C57BL/6 mice were administered 2DN (15 mg/kg) or saline alone, and after 24 h, were re-injected with same. After 18 h, mice were sacrificed and lungs harvested and assayed for sialidase activity for the fluorogenic substrate, 4-MU-NANA. Vertical bars represent mean±SE sialidase activity in arbitrary fluorescence units. *, significantly decreased sialidase activity compared to absence of 2DN at p<0.05. Represents ≥4 independent experiments. (B-E) Bleomycin-challenged mice were daily administered 2DN (15 mg/kg BW) or vehicle, alone on days 8-14 and sacrificed on day 14. (B) Mice were serially weighed over the 14-day study period. Each symbol represents mean±SE percent of initial pretreatment total body weight. (C) On day 14, BALF was collected and processed for total and differential cell counts. Each vertical bar represents mean±SE BALF cell count×10⁵. (D-E) Lungs were harvested and processed for qRT-PCR for collagen mRNA expression (D), or homogenized for hydroxyproline-based assays for quantitation of collagen protein (E). In (D), each vertical bar represents mean±SE mRNA for either collagen 1A2 or collagen 3A1 normalized to 18s rRNA internal control. In (E), each symbol represents μg collagen protein/mg wet lung tissue. (B-E) *, indicates significantly increased compared to the vehicle control at p<0.05. **, indicates significantly decreased compared to bleomycin in absence of 2DN at p<0.05.

FIG. 2. NEU and PPCA Expression in the Lung Tissues of Bleomycin-Challenged Mice. C57BL/6 mice were administered bleomycin or vehicle alone, and after 14 days, were sacrificed and lungs harvested. (A) Total RNA isolated from lung tissues was reverse-transcribed, and the resulting cDNA was used as a template for amplification with primers corresponding to NEU1, NEU2, NEU3, NEU4, and PPCA. The mRNA levels for each NEU and PPCA were normalized to the 18S rRNA internal control. Each vertical bar represents mean±SE normalized mRNA levels. (B) Lungs were homogenized and the homogenates processed for NEU1, NEU2, NEU3, and NEU4 immunoblotting. To control for protein loading and transfer, blots were stripped and reprobed for GAPDH. IB, immunoblot; IB*, immunoblot after stripping. MW in kDa is indicated on the left. Each blot is representative of 3 independent experiments. In the case of NEU1, densitometric analyses of the NEU1 immunoblots are included. Vertical bars represent mean±SE NEU1 signal normalized to GAPDH signal in the same lane on the same stripped and reprobed blot. *, significantly increased normalized NEU1 mRNA/protein in bleomycin-challenged mice compared to vehicle-treated controls at p<0.05.

FIG. 3. Effect of NEU1-Selective Pharmacologic Blockade on Sialidase Activity in Human Lung Cells in vitro. SAECs (A), HPMECs (B), and HLFs (C) were each assayed for total sialidase activity at 1.0×10⁶ cells/reaction in the presence of increasing concentrations of the NEU1-selective inhibitor, III-32B5. Vertical bars represent mean±SE sialidase activity expressed as arbitrary fluorescence units per 10⁶ cells.

FIG. 4. NEU1-Selective Sialidase Inhibition Protects Against Lymphocyte Infiltration and Collagen Deposition in the Lungs of Bleomycin-Challenged Mice. Bleomycin-challenged C57BL/6 mice were daily administered equivalent doses of C9-BA-DANA or III-32B5 (15 mg/kg BW) or vehicle alone on days 8-14 and sacrificed on day 14. (A+D) Mice were serially weighed over the 14-day study period. Each symbol represents mean±SE percent of initial pretreatment total body weight. (B+E) On day 14, BALF was collected and processed for total and differential cell counts. Each vertical bar represents mean±SE BALF cell count×10⁵. (C+F) Lungs were harvested and processed for hydroxyproline-based assays to quantitate collagen protein. Each symbol represents μg collagen/mg wet lung tissue. *, indicates significantly increased compared to vehicle control at p<0.05. **, indicates significantly decreased compared to bleomycin in absence of either C9-BA-DANA or III-32B5 at p<0.05.

FIG. 5. The compound C9-BADANA.

FIG. 6. NEU1 Inhibition Protects the Lungs of Mice from Bleomycin-Induced Fibrosis. Mice were challenged with BLM (day 0), then daily given i.p. C9-BADANA, III-32B5, or saline on days 8 through 14. A and B: At day 14, collagen (scar material) was low in naive mice (PBS, black circles), very high in BLM mice (red circles), and significantly decreased in BLM mice treated with NEU1 inhibitors (blue circles). **, significantly decreased at p<0.05 compared to BLM alone. C and D: Lung fibrosis is associated with decreased body weight (red lines). It is counteracted by NEU1 inhibition (blue lines). SEM shown.

FIG. 7. BLM Upregulates NEU1 Expression in the Lung. Mice given BLM or vehicle (PBS) were sacrificed on day 14, and qRT-PCR was performed on lung tissue. NEU1 mRNA normalized to 18S RNA increased >fourfold in the experimental group. *, p>0.05.

### DETAILED DESCRIPTION

As described herein, we have discovered that NEU1-selective inhibition provides a therapeutic intervention for pulmonary fibrosis in an animal model of (bleomycin-induced) pulmonary fibrosis. Accordingly, provided herein are methods for treating or preventing fibrotic lung diseases and/or conditions involving increased NEU1 activity. NEU1 inhibitors (for example, NEU1-selective inhibitors) can be administered to patients in need thereof to treat or prevent profibrotic diseases of the lung. Such patients include those diagnosed with, or are at risk of, developing a pulmonary fibrotic disease and/or conditions. Such patients include those with idiopathic pulmonary fibrosis (IPF), patients with connective tissue diseases and/or autoimmune diseases in which there is a risk of developing lung fibrosis, including scleroderma, rheumatoid arthritis, and patients in which there is fibrosis-inducing damage to the lung tissue. Such fibrosis-inducing damage can include medical treatments such as chemotherapy and/or radiation therapy for cancer. Other causes of fibrosis-inducing damage that can be treated by the methods of the invention include accidental or deliberate exposure to noxious chemicals and/or radiation, such as during warfare, terrorist attacks, or industrial accidents.

To address whether elevated NEU1 expression may contribute to IPF pathogenesis, the impact of forced NEU1 overexpression via gene therapy on primary human lung cell phenotype in vitro was studied. In small airway epithelial cells, NEU1 overexpression restrained cell migration in wounding assays. In microvascular endothelia, NEU1 overexpression increased their ability to capture lymphocytes in a dynamic flow through system, and disrupted capillary-like tube formation on a Matrigel substrate. Finally, in fibroblasts, NEU1 overexpression increased collagen expression at both the mRNA and protein levels. We then asked whether these NEU1-driven functional changes could be extended to an in vivo system. NEU1 overexpression in the murine lung via intratracheal infection with Ad-NEU1 dramatically increased pulmonary recruitment of lymphocytes, with perivascular, peribronchial, and patchy parenchymal infiltrates, as well as increased CD8+ T cells in the BALF. In these same NEU1-overexpressing lung tissues, Masson's trichrome staining revealed increased college deposition. Quantitative hydroxyproline-based measurements and immunoblotting for Type I collagen of lung homogenates confirmed increased abundance of collagen. Taken together, each of these NEU1-provoked phenotypic changes, including impaired airway epithelial wound healing, disrupted pulmonary microvascular angiogenesis, increased lymphocyte infiltration of lung tissues, and increased fibroblast collagen biosynthesis and deposition, could contribute to the IPF disease state.

Since NEU1 expression was clearly elevated in the lung tissues of IPF patients, and forced NEU1 overexpression in multiple human lung cells in vitro, and murine lungs in vivo, provoked phenotypic changes commonly associated with IPF, we asked whether elevated NEU1 catalytic activity might be causally linked to the increased lymphocyte infiltration and collagen deposition seen in fibrotic lung tissues, and whether the NEU1 sialidase might offer a target for therapeutic intervention. To address these questions, as described herein, we tested whether broad-spectrum and NEU1-selective sialidase inhibition can protect against pulmonary fibrosis in bleomycin-challenged mice.

Reference will now be made in detail to embodiments of the invention which, together with the drawings and the following examples, serve to explain the principles of the invention. These embodiments describe in sufficient detail to enable those skilled in the art to practice the invention, and it is understood that other embodiments may be utilized, and that structural, biological, and chemical changes may be made without departing from the spirit and scope of the present invention. Unless defined otherwise, all technical and scientific terms used herein have the same meanings as commonly understood by one of ordinary skill in the art.

For the purpose of interpreting this specification, the following definitions will apply and whenever appropriate, terms used in the singular will also include the plural and vice versa. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used). The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. Furthermore, where the description of one or more embodiments uses the term “comprising,” those skilled in the art would understand that, in some specific instances, the embodiment or embodiments can be alternatively described using the language “consisting essentially of” and/or “consisting of.” As used herein, the term “about” means at most plus or minus 10% of the numerical value of the number with which it is being used.

It is contemplated that any method or composition described herein can be implemented with respect to any other method or composition described herein.

One skilled in the art may refer to general reference texts for detailed descriptions of known techniques discussed herein or equivalent techniques. These texts include Current Protocols in Molecular Biology (Ausubel et. al., eds. John Wiley & Sons, N.Y. and supplements thereto), Current Protocols in Immunology (Coligan et al., eds., John Wiley St Sons, N.Y. and supplements thereto), Current Protocols in Pharmacology (Enna et al., eds. John Wiley & Sons, N.Y. and supplements thereto) and Remington: The Science and Practice of Pharmacy (Lippincott Williams & Wilicins, 2Vt edition (2005)), for example.

“Fibrosis” is a lesion characterized by the activation and proliferation of fibroblasts, and increased fibrous connective tissue and decreased parenchymal cells in tissues and organs, and structural destruction and loss of function of tissues and organs, after the tissues and organs such as lung, liver, kidney, blood vessels, peritoneum, pancreas and/or skin are continuously injured due to various causes such as inflammation, infection, immune response, ischemia, chemicals and/or radiation. The term can be used interchangeably with “fibrotic lesion”. The term “fibrotic lesion” encompasses fibrotic lesions in tissues and organs, such as cardiac fibrosis, pulmonary fibrosis, hepatic fibrosis, renal fibrosis, vascular fibrosis and skin fibrosis, which are caused by various factors, and further comprises the fibrotic lesions in tissues and organs, such as cardiac fibrosis, pulmonary fibrosis, hepatic fibrosis, renal fibrosis, vascular fibrosis and skin fibrosis, which are associated with the development and progression of various diseases.

“Pulmonary fibrosis” refers to a pathological process caused by lung tissue mesenchymal cell proliferation, extracellular matrix proliferation and deposition, and lung parenchymal remodeling which are caused or accompanied by various factors (such as inflammation, infection, immune response, ischemia, chemicals, and/or radiation). Pulmonary fibrosis lesions lead to impaired pulmonary function, and thus the resulting related conditions are called “pulmonary fibrosis-related conditions.”

As used herein, the terms “inhibit” and “inhibition” mean to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels. For example, “inhibits” means hindering, interfering with or restraining the activity of the gene relative to a standard or a control. “Inhibits” can also mean to hinder or restrain the synthesis, expression or function of the protein relative to a standard or control.

The terms “antagonist” and “antagonistic” as used herein refer to or describe an agent that is capable of, directly or indirectly, partially or fully blocking, inhibiting, reducing, or neutralizing a biological activity of a target and/or pathway. The term “antagonist” is used herein to include any agent that partially or fully blocks, inhibits, reduces, or neutralizes the activity of a protein.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus those in need of treatment include those already with the disorder; those prone to have the disorder; and those in whom the disorder is to be prevented.

The terms “modulation” and “modulate” as used herein refer to a change or an alteration in a biological activity. Modulation includes, but is not limited to, stimulating an activity or inhibiting an activity. Modulation may be an increase or a decrease in activity, a change in binding characteristics, or any other change in the biological, functional, or immunological properties associated with the activity of a protein, a pathway, a system, or other biological targets of interest.

The term “binding” refers to the interaction between a corresponding pair of molecules or portions thereof that exhibit mutual affinity or binding capacity, typically due to specific or non-specific binding or interaction, including, but not limited to, biochemical, physiological, and/or chemical interactions. “Binding partner” or “ligand” refers to a molecule that can undergo specific binding with a particular molecule. “Biological binding” defines a type of interaction that occurs between pairs of molecules including proteins, peptides, nucleic acids, glycoproteins, carbohydrates, or endogenous small molecules. “Specific binding” refers to molecules, such as polynucleotides, that are able to bind to or recognize a binding partner (or a limited number of binding partners) to a substantially higher degree than to other, similar biological entities.

As used herein, the term “specifically binds” refers to the binding of a molecule, e.g., a drug, to a protein molecule, while not significantly binding to other protein molecules. In some embodiments, an agent “specifically binds” to a target molecule with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more).

As used herein, the term “monoclonal antibody” or “MAb” refers to an antibody obtained from a substantially homogeneous population of antibodies, i.e., the individual antibodies within the population are identical except for possible naturally occurring mutations that may be present in a small subset of the antibody molecules.

The term “antibody” refers to natural or synthetic antibodies that bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that bind the target antigen. Thus, the term “antibody” encompasses a molecule having at least one variable region from a light chain immunoglobulin molecule and at least one variable region from a heavy chain molecule that in combination form a specific binding site for the target antigen. The antibody can be a IgG antibody, for example, the antibody can be a IgG1, IgG2, IgG3, or IgG4 antibody.

An “antibody fragment” or “antigen binding fragment” of an antibody is defined as at least a portion of the variable region of the immunoglobulin molecule that binds to its target, i.e., the antigen-binding region. An antibody can be in the form of an antigen binding antibody fragment including a Fab fragment, F(ab′)₂ fragment, a single chain variable region, and the like. Fragments of intact molecules can be generated using methods well known in the art and include enzymatic digestion and recombinant means.

As used herein, the term “single chain Fv” or “scFv” as used herein means a single chain variable fragment that includes a light chain variable region (V_{L}) and a heavy chain variable region (V_{H}) in a single polypeptide chain joined by a linker which enables the scFv to form the desired structure for antigen binding (i.e., for the V_{H} and V_{L} of the single polypeptide chain to associate with one another to form a Fv). The V_{L} and V_{H} regions may be derived from the parent antibody or may be chemically or recombinantly synthesized.

The term “variable region” is intended to distinguish such domain of the immunoglobulin from domains that are broadly shared by antibodies (such as an antibody Fc domain) The variable region includes a “hypervariable region” whose residues are responsible for antigen binding. The hypervariable region includes amino acid residues from a “Complementarity Determining Region” or “CDR” (i.e., typically at approximately residues 24-34 (L), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., residues 26-32 (L), 50-52 (L2) and 91-96 (L3) in the light chain variable domain and 26-32 (H1), 53-55 (H2) and 96-101 (H3) in the heavy chain variable domain; Chothia and Lesk, 1987, J. Mol. Biol. 196:901-917). The term “Framework Region” or “FR” residues are those variable domain residues other than the hypervariable region residues as herein defined.

A “neutralizing antibody”, (or an “antibody that neutralizes NEU1 activity”), is intended to refer to an antibody whose binding to NEU1 results in inhibition of the biological activity of NEU1. This inhibition of the biological activity of NEU1, or its ligands, can be assessed by measuring one or more indicators of NEU1 biological activity. These indicators of NEU1 biological activity can be assessed by one or more of several standard in vitro or in vivo assays known in the art.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention, for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies or other members of the immunoglobulin superfamily, in certain embodiments, a “polypeptide” can occur as a single chain or as two or more associated chains.

The terms “polynucleotide” and “nucleic acid” and “nucleic acid molecule” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

As used herein, the term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 nucleotides or amino acid residues, at least about 60-80 nucleotides or amino acid residues in length or any integral value there between. In some embodiments, identity exists over a longer region than 60-80 nucleotides or amino acid residues, such as at least about 80-100 nucleotides or amino acid residues, and in some embodiments the sequences are substantially identical over the full length of the sequences being compared, for example, the coding region of a nucleotide sequence.

A “conservative amino acid substitution” is one in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Generally, conservative substitutions in the sequences of polypeptides and/or antibodies of the invention do not abrogate the binding of the polypeptide or antibody containing the amino acid sequence, to the target binding site. Methods of identifying nucleotide and amino acid conservative substitutions which do not eliminate binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, soluble protein, antibody, polynucleotide, vector, cell, or composition which is “isolated” is a polypeptide, soluble protein, antibody, polynucleotide, vector, cell, or composition which is in a form not found in nature. Isolated polypeptides, soluble proteins, antibodies, polynucleotides, vectors, cells, or compositions include those which have been purified to a degree that they are no longer in a form in which they are found in nature. In some embodiments, a polypeptide, soluble protein, antibody, polynucleotide, vector, cell, or composition which is isolated is substantially pure.

The term “subject” refers to any animal (e.g., a mammal), including, but not limited to, humans, non-human primates, canines, felines, rabbits, rodents, and the like, which is to be the recipient of a particular treatment. Typically, the terms “subject” and “patient” are used interchangeably herein in reference to a human subject.

The term “pharmaceutically acceptable” refers to a substance approved or approvable by a regulatory agency of the Federal government or a state government or listed in the U.S. Pharmacopeia or other generally recognized pharmacopeia for use in animals, including humans.

The terms “pharmaceutically acceptable excipient, carrier, or adjuvant” or “acceptable pharmaceutical carrier” refer to an excipient, carrier, or adjuvant that can be administered to a subject, together with at least one agent of the present disclosure, and which does not destroy the pharmacological activity thereof and is non-toxic when administered in doses sufficient to deliver a therapeutic effect. In general, those of skill in the art and the U.S. FDA consider a pharmaceutically acceptable excipient, carrier, or adjuvant to be an inactive ingredient of any formulation.

The terms “effective amount” or “therapeutically effective amount” or “therapeutic effect” refer to an amount of an agent described herein, an antibody, a polypeptide, a polynucleotide, a small organic molecule, or other drug effective to “treat” a disease or disorder in a subject such as, a mammal.

The term “and/or” as used in a phrase such as “A and/or B” herein is intended to include both A and B; A or B; A (alone); and B (alone). Likewise, the term “and/or” as used in a phrase such as “A, B. and/or C” is intended to encompass each of the following embodiments: A. B, and C; A, B. or C; A or C; A or B; B or C; A and C; A and B; B and C; A (alone), B (alone); and C (alone).

### Methods

In one embodiment, the invention provides a method for treating a fibrotic lung disease or condition in a subject that involves an increase in NEU1 expression and/or activity, comprising administering to the subject an effective amount of an agent that inhibits the activity of NEU1 sialidase, thereby treating the fibrotic lung disease or condition in the subject.

In some embodiments, fibrotic lung diseases or conditions comprise idiopathic pulmonary fibrosis, sarcoidosis, allergic pneumonia, pneumoconiosis, drug-induced and radiation-induced fibrosis, and a broad spectrum of diseases with varying etiologies such as fibrogenic alveolitis associated with collagen vascular disease. The main pathological features can comprise lung tissue mesenchymal cell proliferation, extracellular matrix proliferation and deposition, and remodeling of lung parenchyma. At present, anti-inflammation, anti-oxidation, anti-fibroblast proliferation, anti-collagen deposition, lung transplantation and other measures are mainly used to treat pulmonary fibrosis.

In some embodiments, the fibrotic lung disease or condition is idiopathic pulmonary fibrosis. In some embodiments, the fibrotic lung disease or condition is associated with a connective tissue disorder. In some embodiments, the fibrotic lung disease or condition is selected from the group consisting of sarcoidosis, allergic pneumonia, pneumoconiosis, drug-induced fibrosis, radiation-induced fibrosis, noxious chemical compound-induced fibrosis, and fibrogenic alveolitis associated with collagen vascular disease. In some embodiments, the agent reduces or prevents myofibroblast accumulation in the subject. In some embodiments, the agent reduces dyspnea caused by the fibrotic lung disease or condition in the subject.

Progressive fibrosis is a hallmark of aging in various organ systems, including the liver, kidney, pancreas and lung. Idiopathic pulmonary fibrosis (IPF) is the most fatal and progressive fibrotic lung disease. It disproportionately affects the elderly population and is now widely regarded as a disease of aging. The incidence and prevalence of IPF increase with age; two-thirds of IPF patients are older than 60 years at the time of presentation with a mean age of 66 years at the time of diagnosis. Further, the survival rate for IPF patients markedly decreases with age.

Idiopathic pulmonary fibrosis is a specific subgroup of pulmonary fibrosis. IPF is a lung disease that results in scarring (fibrosis) of the lungs for an unknown reason. Over time, the scarring gets worse and it becomes hard to take in a deep breath and the lungs cannot take in enough oxygen. IPF is a form of interstitial lung disease, primarily involving the interstitium (the tissue and space around the air sacs of the lungs), and not directly affecting the airways or blood vessels. The cause of idiopathic pulmonary fibrosis is not completely understood.

Common risk factors for IPF include genetic background, with up to 20% of people with IPF having another family member with an interstitial lung disease. Where more than one additional family member has IPF, the disease is termed “familial pulmonary fibrosis.”

Cigarette smoking is another factor, with approximately 75% of people with IPF being current or previous cigarette smokers. Acid reflux (gastroesophageal reflux disease [GERD]) is also another factor, with approximately 75% of people with IPF having symptoms of acid reflux (heartburn). Male sex is another risk factor, with approximately 75% of patients with IPF being male. Age is also important, with almost all patients with IPF are over the age of 50 years.

Clinical signs of IPF indicative of a need for treatment include any one or more of dyspnea (i.e., breathlessness, shortness of breath), usually during exercise, chronic cough, chest pain or tightness, unexplained weight loss, loss of appetite, fatigue, and clubbing of the digits (i.e., change of finger shape).

About 85% of people with IPF have a chronic cough that has lasts longer than 8 weeks. This is often a dry cough, but some people may also cough up sputum or phlegm. Breathlessness can affect day-to-day activities such as showering, climbing stairs, getting dressed and eating. As scarring in the lungs gets worse, breathlessness may prevent all activities.

Methods for identifying a subject having IPF are known in the art, Exemplary clinical diagnostic techniques include pulmonary function test (PFT; or breathing test) to measure how much air can be inhaled/exhaled blow in and out of your lungs and capacity for lungs to absorb oxygen; six-minute walk test to determine physical fitness, as well as the amount of oxygen in the blood at rest and with physical activity; chest x-ray: Chest X to screen for interstitial lung disease and to monitor progression; blood tests for serological identity of other causes of interstitial lung disease; computed tomography (CT scan) to determine extent of scarring in the lungs; bronchoscopy to identify the presence of infection or to suggest other subtypes of interstitial lung disease; and surgical lung biopsy.

Signs of the improvement in pulmonary fibrosis, for example, in response to treatment with one or more inhibitors of NEU1, include an improvement in any one or more of the above symptoms.

Criteria constituting treatment failure in PF include any worsening/no change of the above symptoms, side effects such as issues with toxicity/tolerability/drug-drug interactions with drugs patient already taking, infections due to administration issues, worsening or no change in an observable factor such as 6 minute walk distance, and worsening or no change in cardiopulmonary test results (e.g., worsening oxygen consumption).

For the treatment of a disease, the appropriate dosage of an agent that inhibits the activity of NEU1 of the present invention depends on the type of disease to be treated, the severity and course of the disease, the responsiveness of the disease, whether the agent is administered for therapeutic or preventative purposes, previous therapy, the patient's clinical history, and so on, all at the discretion of the treating physician. The agent that inhibits the activity of NEU1 can be administered one time or over a series of treatments lasting from several days to several months, or until a cure is effected or a diminution of the disease state is achieved. Optimal dosing schedules can be calculated from measurements of drug accumulation in the body of the patient and will vary depending on the relative potency of an individual agent. The administering physician can determine optimum dosages, dosing methodologies, and repetition rates. In certain embodiments, dosage is from 0.01 μg to 100 mg/kg of body weight, from 0.1 μg to 100 mg/kg of body weight, from 1 μg to 100 mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In certain embodiments, the dosage of the agent is from about 0.1 mg to about 20 mg/kg of body weight. In some embodiments, the dosage of the agent is about 0.5 mg/kg of body weight. In some embodiments, the dosage of the agent is about 1 mg/kg of body weight. In some embodiments, the dosage of the agent is about 1.5 mg/kg of body weight. In some embodiments, the dosage of the agent is about 2 mg/kg of body weight. In some embodiments, the dosage of the agent is about 2.5 mg/kg of body weight. In some embodiments, the dosage of the agent is about 5 mg/kg of body weight. In some embodiments, the dosage of the agent is about 7.5 mg/kg of body weight. In some embodiments, the dosage of the agent is about 10 mg/kg of body weight. In some embodiments, the dosage of the agent is about 12.5 mg/kg of body weight. In some embodiments, the dosage of the agent is about 15 mg/kg of body weight. In certain embodiments, the dosage can be given once or more daily, weekly, monthly, or yearly. In certain embodiments, the agent is given once every week, once every two weeks, once every three weeks, or once every four weeks.

In some embodiments, an agent that inhibits the activity of NEU1 may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

In some embodiments, the dosing schedule may be limited to a specific number of administrations or “cycles”. In some embodiments, the agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art.

In some embodiments, the invention provides methods of administering to a subject an agent that inhibits the activity of NEU1 described herein comprising using an intermittent dosing strategy for administering one or more agents, which may reduce side effects and/or toxicities associated with administration of an agent. In some embodiments, the method comprises administering to the subject a therapeutically effective dose of an agent wherein the agent is administered according to an intermittent dosing strategy. In some embodiments, the intermittent dosing strategy comprises administering an initial dose of an agent to the subject, and administering subsequent doses of the agent about once every 2 weeks. In some embodiments, the intermittent dosing strategy comprises administering an initial dose of an agent to the subject, and administering subsequent doses of the agent about once every 3 weeks. In some embodiments, the intermittent dosing strategy comprises administering an initial dose of an agent to the subject, and administering subsequent doses of the agent about once every 4 weeks.

### NEU1 Inhibitors

Blockade of the NEU1 expression and/or function of NEU1 can reduce or prevent processes that give rise to the onset and development of fibrotic lung diseases or conditions. Agents that inhibit or reduce the transcription, translation or function of the NEU1 enzyme are described.

In some embodiments, an agent that inhibits the activity of NEU1 acts by modulating expression of NEU1 or one or more components of a NEU1 containing complex. The expression can be at the mRNA level, protein level or both. In some embodiments, the agent modulates the expression by reducing or inhibiting expression of NEU1 and/or one or more components of a NEU1 containing complex.

In some embodiments, the agent is NEU1 selective and binds the NEU1 catalytic domain.

In some embodiments, an agent that inhibits the activity of NEU1 acts as an antagonist of NEU1 or a NEU1 containing complex. In some embodiments, the NEU1 containing complex comprises TOLL-like receptor. In some embodiments, NEU1 is localized to lysosomes where it resides in a multienzyme complex, with protective protein/cathepsin A (PPCA) and β-galactosidase. It has been disclosed that ligand-induced TOLL-like receptor (TLR) activation is controlled by NEU1 sialidase activation. Studies have shown that NEU1 is already in complex with the TOLL-like receptors, and activation is induced upon ligand binding of the natural ligands to their respective receptors. In addition, activated NEU1 specifically hydrolyzes α-2,3-sialyl residues linked to β-galactoside, which are distant from the ligand binding site. This removes steric hindrance to receptor dimerization, and leads to subsequent signalling pathways. Amith et al. Glycoconj J 2009, 26, 1197-1212; Amith et al. Cell Signal 2010, 22, 314-324.

In some embodiments, the agent binds to NEU1 and inhibits its activity.

In some embodiments, the agent disrupts the interaction between NEU1 and one or more components of a NEU1-containing complex.

In some embodiments, the agent disrupts the interaction between one or more components of the NEU1 containing complex with one or more other components of the NEU1-containing complex.

Agents that inhibit NEU1 can bind to the NEU1 gene or to NEU1 polypeptide and directly or indirectly block the biological function of NEU1 polypeptide. Inhibitors can also block the biological function of one or more signaling pathways that constitute the down-stream biological function of NEU1. In some embodiments, the inhibitors can block protein-protein interactions involving the NEU1 polypeptide, or they can prevent or reduce the functional activity of a complex of the NEU1 enzyme and a receptor. Inhibitors that bind directly to the NEU1 polypeptide may act by direct occlusion of an active site on the NEU1 polypeptide, or through indirect occlusion, such as by stearic blockade of NEU1 interactions. For example, in some embodiments the inhibitor obstructs or occludes the function of a protein domain, such as the enzyme active site. In other embodiments, inhibitors bind to a location that is spatially distinct from an active site. Therefore, in certain embodiments, inhibitors that bind to the NEU1 polypeptide can prevent NEU1 function by mechanisms including, but not limited to, inducing conformational changes, prevent catalytic functions, inducing degradation, inducing uptake by immune cells, preventing uptake by target cells, preventing ligand binding, preventing phosphorylation, inducing denaturation, preventing one or more post-translational modifications or otherwise altering the native tertiary structure of the NEU1 polypeptide.

It is understood that initiation or transduction of cellular signaling pathways by NEU1 can require binding of a receptor by the NEU1 polypeptide. Therefore, proteins, antibodies or small molecules that block signal transduction pathways involving NEU1 and optionally prevent co-ligation of NEU1 and its receptors are useful agents. Classes of NEU1 inhibitors discussed below include antibodies, Fab fractions of antibodies and functional nucleic acids that bind directly to the NEU1 polypeptide, as well as antibodies, Fab fractions of antibodies and functional nucleic acids that bind to ligands of NEU1.

### Small Molecule Inhibitors

Small molecules that specifically inhibit the transcription, translation or function of the NEU1 gene and/or gene product are described. In some embodiments, the small molecule inhibitors of NEU1 are non-protein, non-nucleic acid molecules that have a specific function, such as binding a target molecule or reducing, preventing or otherwise moderating a specific reaction or interaction. As discussed in more detail below, the term “small molecules” generally include a molecule of less than 10,000 Da in molecular weight. Small molecules that specifically interact with NEU1 or associated proteins can act as effectors, inhibitors, modulators, and stimulators of a specific activity possessed by a target molecule, or the small molecules can possess a de novo activity independent of any other molecules. Preferred small molecule inhibitors of NEU1 have excellent dose-dependent enzyme inhibitory properties.

In some embodiments, the agent is a small inhibitory peptide to inhibit the function of NEU1. In some embodiments, the peptide is formulated in a nanoparticle for delivery to the cell.

In some embodiments, the NEU1 inhibitor can include, for example, a broad-spectrum sialidase inhibitor, such as 2-deoxy-N-acetyneuraminic acid (2DN).

In some embodiments, the NEU1 inhibitor can include, for example, any of the compounds described in WO 2018/213933, which is herein incorporated by reference in its entirety. In some embodiments, the NEU1 inhibitor is any of the compounds described in Guo et al., J. Med. Chem. 2018, 61:11261-11279 (which is incorporated by reference in its entirety), including compound 17f therein, corresponding to compound III-32B5 (also known as compound CG33300 or CG33301 for its methyl ester). The chemical name for compound III-32B5 is C5-hexanamido-C9-acetamido-DANA. The chemical structure of compound III-32B5 (PubChem ID 145989099 and CHEMBL4290253) is shown below:

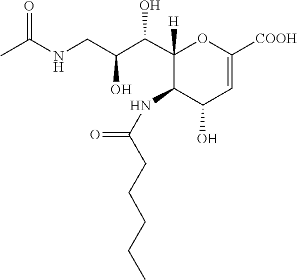 [SMILES: CCCCCC(=O)N[C@H]1[C@H]([C@@H](O)[C@@H](O)CNC(C)=O)OC(C(=O)O)=C[C@@H]1O]

Esters, solvates, hydrates or pharmaceutical salts are also included. In some embodiments, the NEU1 inhibitors comprise C1-methyl esters of these compounds.

In some embodiments, the NEU1 inhibitor comprises a compound of formula (I):

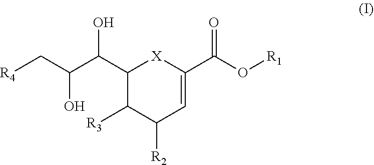

wherein R₁ is H; a C1-C10 alkyl; C1-C10 heteroalkyl; C3-C7 cycloalkyl; C3-C7 heterocycloalkyl; or C3-C8 aryl; or C3-C8 heteroaryl; wherein the alkyl, heteroalkyl, cycloalkyl, heterocycloalkyl, aryl and heteroaryl are optionally substituted by at least one substituent, each substituent being independently a C1-C10 alkyl, a C3-C8 cycloalkyl, a C3-C7 aryl, an halogen, an amide or an hydroxyl;

R₂ is H; —OH, —NHC(═NH)NH₂; or azide;

R₃ is —NHC(O)(CH₂)nR₅,

wherein R₅ is H; —OH; C1-C10 alkyl; C1-C10 heteroalkyl; C3-C7 cycloalkyl; C3-C7 heterocycloalkyl; or C3-C8 aryl; wherein the alkyl, heteroalkyl, cycloalkyl, heterocycloalkyl, and aryl are optionally substituted by at least one substituent, each substituent being independently a C1-C10 alkyl, a C3-C8 cycloalkyl, a C3-C7 aryl, an halogen, an amide or an hydroxyl; and

n is 0 or 1;

R₄ is H; —OH; —O-alkyl; —NHC(O)R₆; or

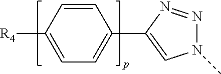

wherein R₆ is H, C1-C10 alkyl; or C3-C7 aryl;

R₇ is H; halogen; —O-alkyl; —C(O)OH; amine; acetamide; —C1-C10 alkyl; —O—C3-C7 aryl; or —(CH₂)qNH(CO)aryl; p is 0, 1, 2 or 3; and

q is 0 or 1; and

X is 0, CH₂ or S,

with the proviso that when R₂ and R₄ are OH, R₃ is not-NHC(O)CH₃,

or is an ester, solvate, hydrate or pharmaceutical salt thereof.

In some embodiments, the NEU1 inhibitors comprise esters of these compounds, including Cl-methyl esters.

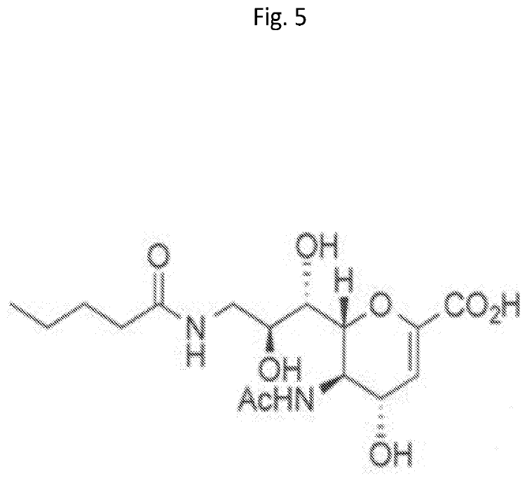

In some embodiments, the NEU1 inhibitor is the compound C9-BADANA shown below (see also FIG. 5):

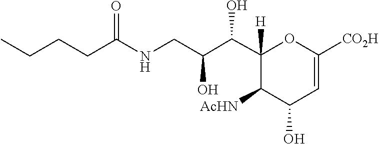 [SMILES: CCCCC(=O)NC[C@H](O)[C@H](O)[C@@H]1OC(C(=O)O)=C[C@H](O)[C@H]1NC(C)=O]

C9-BADANA is described in Magesh et al., Bioorg Med Chem Lett, 18:532-537, 2008 and Hyun et al. Glycobiology 26:834-849, 2016, which are incorporated by reference in their entireties. Esters, solvates, hydrates or pharmaceutical salts are also included. In some embodiments, the NEU1 inhibitor is a C1-methyl ester of this compound.

In some embodiments, the NEU1 inhibitor is selected from oseltamivir phosphate, BCX-1827, DANA (2-deoxy-2,3-dehydro-N-acetylneuraminic acid), zanamivir (4-guanidino-Neu5Ac2en), and oseltamivir carboxylate.

The inhibitors can be administered alone or in combination by any appropriate route of administration as will be understood by one of ordinary skill in the art. Such systemic routes of administration can include oral, parenteral, and intravenous. Appropriate local routes of administration (e.g., to the lung) can include via inhalation, as will be understood by the skilled artisan.

### Antibodies

Antibodies that inhibit the function of NEU1 by specific interaction directly with the NEU1 enzyme, its binding partners, or its accessory molecules can be used. Antibodies can include an antigen binding site that binds to an epitope on the NEU1 enzyme. Binding of an antibody to NEU1 can inhibit or reduce the function of the NEU1 enzyme via one or more distinct mechanisms. Typically, the antibodies can reduce or neutralize NEU1 biological activity in vitro and in vivo. In some embodiments, the antibodies have high affinity for NEU1 (e.g., K_{d}=10⁻⁸ M or less), a slow off rate for NEU1 dissociation (e.g., K_{off}=10⁻³ sec⁻¹, or less), or a combination thereof.

Full-length antibodies, antigen binding fragments thereof, and fusion proteins based thereon are provided. Useful antibodies, and antigen-binding fragments thereof are typically characterized by binding to NEU1, or one or more ligands of NEU1, preferably with high affinity and slow dissociation kinetics. In some embodiments, the antibodies, or antigen-binding fragments thereof inhibit NEU1 activity. The antibodies can be full-length (e.g., an IgG subtype 1, or IgG4 antibody) or can comprise only an antigen-binding portion (e.g., a Fab, F(ab′)2′ scFv fragment, or F(Ab) single domain).

In some embodiments, inhibitors of NEU1, or ligands of NEU1, are proteins that have the antigen-binding specificity of an antibody, such as a fragment of an antibody. The term “antigen-binding portion” of an antibody (or simply “antibody portion”), refers to one or more fragments of an antibody that retain the ability to specifically bind to an antigen (e.g., NEU1).

Various types of antibodies and antibody fragments can be used in the disclosed compositions and methods, including whole immunoglobulin of any class, fragments thereof, and synthetic proteins containing at least the antigen binding variable domain of an antibody. The antibody can be an IgG antibody, such as IgG₁, IgG₂, IgG₃, or IgG₄. An antibody can be in the form of an antigen binding fragment including a Fab fragment, F(ab′)2 fragment, a single chain variable region, and the like. Antibodies can be polyclonal or monoclonal (mAb).

Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison, et al., Proc. Natl. Acad. Sci. USA, 81: 6851-6855 (1984)).

The antigen-binding function of an antibody can be performed by fragments of a full-length antibody. Examples of binding fragments encompassed within the term “antigen binding portion” of an antibody include (i) a Fab fragment, a monovalent fragment consisting of the VL, VH, CL and CH domains; (ii) a F(ab′)2 fragment, a bivalent fragment comprising two Fab fragments linked by a disulfide bridge at the hinge region; (iii) a Fd fragment consisting of the VH and CH domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites.

A linear epitope is an epitope that formed by a continuous sequence of amino acids from the antigen. Linear epitopes typically include approximately 5 to about 10 continuous amino acid residues. Antibodies bind a linear epitope based on the primary sequence of the antigen. Thus, in some embodiments, the epitope can be a linear epitope and can include 1, 2, 3, 4, 5, 6, 7, 8, 9, 10, or more consecutive amino acids of the primary sequence of SEQ ID NO: 2. A “conformational epitope” is an epitope that includes discontinuous sections of the antigen's amino acid sequence. Antibodies bind a conformational epitope based on 3-D surface features, shape, or tertiary structure of the antigen. Thus, in some embodiments, the antibody or antigen binding fragment thereof can bind a conformational epitope that includes a 3-D surface feature, shape, or tertiary structure of the NEU1 enzyme. In some embodiments, a 3-D surface feature can include any number of amino acids from SEQ ID NO: 2, or the corresponding residues in a homolog, ortholog, paralog, or variant thereof.

In some embodiments, the antibody or antigen binding fragment that binds specifically to an epitope within the protein encoded by the amino acid sequence of SEQ ID NO: 2 can only bind if the protein encoded by the amino acid sequence of SEQ ID NO: 2 is not bound by a ligand or small molecule.

In some embodiments, the antibody or antigen binding portion thereof dissociates from human NEU1, or a ligand of human NEU1, with a K_{off} rate constant of 1×10⁻¹/s⁻¹ or less. Preferably, the antibody, or antigen-binding portion thereof, dissociates from human NEU1, or a ligand of human NEU1 with a K_{off} rate constant of 5×10⁻⁴/s⁻¹ or less. Even more preferably, the antibody, or antigen binding portion thereof, dissociates from human NEU1, or a ligand of human NEU1 with a K_{off} rate constant of 1×10⁻⁴/s⁻¹ or less or less. Typically, the anti-NEU1 antibody binds an epitope formed by two or more amino acid residues at the surface of the tertiary structure of the NEU1 enzyme formed by the amino acid sequence of SEQ ID NO. 2.

Commercial antibodies specific for NEU1 are available. For example, polyclonal and monoclonal rabbit or mouse anti-human NEU1 antibodies are commercially available from multiple vendors.

In one embodiment, the antibody is a mouse monoclonal antibody (OTI3D4), corresponding to ThermoFisher Scientific Cat #TA801727.

In one embodiment, the antibody is a mouse monoclonal antibody (OTI3B3), corresponding to ThermoFisher Scientific Cat #MA5-26508.

In one embodiment, the antibody is a mouse monoclonal antibody (OTI3C4), corresponding to ThermoFisher Scientific Cat #TA801668.

In one embodiment, the antibody is a mouse monoclonal antibody (688215), corresponding to ThermoFisher Scientific Cat #MA5-24313.

In one embodiment, the antibody is a mouse monoclonal antibody (OTI3H2), corresponding to ThermoFisher Scientific Cat #CF801667.

In one embodiment, the antibody is a mouse monoclonal antibody (OTI3H4), corresponding to ThermoFisher Scientific Cat #CF801669.

In one embodiment, the antibody is a mouse monoclonal antibody (OTI3F5), corresponding to ThermoFisher Scientific Cat #CF801670.

In one embodiment, the antibody is a mouse monoclonal antibody (OTI3B8), corresponding to ThermoFisher Scientific Cat #CF801671.

In one embodiment, the antibody is a mouse monoclonal antibody (OTI3C5), corresponding to ThermoFisher Scientific Cat #CF801703.

In one embodiment, the antibody is a mouse monoclonal antibody (F-8), corresponding to Santa Cruz Biotechnology Cat #sc-166824.

In one embodiment, the antibody is a rabbit polyclonal antibody, corresponding to ThermoFisher Scientific Cat #PA5-42552.

In one embodiment, the antibody is a rabbit polyclonal antibody, corresponding to ThermoFisher Scientific Cat #PA5-54430.

In one embodiment, the antibody is a rabbit polyclonal antibody, corresponding to ThermoFisher Scientific Cat #PA5-53351.

In one embodiment, the antibody is a rabbit polyclonal antibody, corresponding to Sigma Aldrich Product #HPA021506-100UL.

In one embodiment, the antibody is a rabbit polyclonal antibody, corresponding to Sigma Aldrich Product #AV44286-100UG.

In one embodiment, the antibody is a rabbit polyclonal antibody (H-300), corresponding to Santa Cruz Biotechnology Cat #sc-32936.

In some embodiments, a commercially available antibody is used. In some embodiments, the antibody utilized in the disclosed compositions and methods is a humanized or chimeric antibody or an antigen-binding fragment thereof (e.g., a single chain antibody), having one, two, three, four, five, or six CDRs from a commercially available antibody, or having variant CDRs thereof having 65, 70, 75, 80, 85, 90, 95, 96, 97, 98, or 99 percent sequence identity to the corresponding CDRs of commercially available antibody.

In some embodiments, the antibody has the same epitope specificity as a commercially available anti-NEU1 antibody that is otherwise known in the art. This can be achieved by producing a recombinant antibody that contains the paratope of the commercially or otherwise available antibody.

To prepare an antibody that specifically binds to NEU1 or a receptor thereof, purified polypeptides, fragments, fusions, or epitopes thereof, or polypeptides expressed from their nucleic acid sequences, can be used. Using the purified NEU1 or NEU1 ligand polypeptides, or receptor fragments, fusions, or epitopes thereof or proteins expressed from their nucleic acid sequences, antibodies can be prepared using any suitable methods known in the art.

The antibodies can be generated in cell culture, in phage, or in various animals, including mice, rabbits, sheep and horses. Therefore, in some embodiments, an antibody is a mammalian antibody. Phage techniques can be used to isolate an initial antibody or to generate variants with altered specificity or avidity characteristics. Such techniques are routine and well known in the art. In one embodiment, the antibody is produced by recombinant means known in the art. For example, a recombinant antibody can be produced by transfecting a host cell with a vector comprising a DNA sequence encoding the antibody. One or more vectors can be used to transfect the DNA sequence expressing at least one VL and one VH region in the host cell. Exemplary descriptions of recombinant means of antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); Current Protocols In Immunology (John Wiley & Sons, most recent edition).

The antibodies can be modified by recombinant means to increase efficacy of the antibody in mediating the desired function. The antibodies can be modified by substitutions using recombinant means. Typically, the substitutions will be conservative substitutions. For example, at least one amino acid in the constant region of the antibody can be replaced with a different residue. See, e.g., U.S. Pat. Nos. 5,624,821, 6,194,551, Application No. WO 9958572; and Angal, et al., Mol. Immunol. 30:105-08 (1993). The modification in amino acids includes deletions, additions, substitutions of amino acids. In some cases, such changes are made to reduce undesired activities, e.g., complement-dependent cytotoxicity. Frequently, the antibodies are labeled by joining, either covalently or non-covalently, a substance which provides for a detectable signal. A wide variety of labels and conjugation techniques are known and are reported extensively in both the scientific and patent literature. These antibodies can be screened for binding to NEU1 or NEU1 ligand polypeptides, or fragments, or fusions thereof. See e.g., Antibody Engineering: A Practical Approach (Oxford University Press, 1996).

Antibodies that can be used in the disclosed compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each include four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

Also disclosed are fragments of antibodies which have bioactivity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific to an antigenic protein. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

A monoclonal antibody is obtained from a substantially homogeneous population of antibodies, i.e., the individual antibodies within the population are identical except for possible naturally occurring mutations that may be present in a small subset of the antibody molecules. Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

Monoclonal antibodies can be made using any procedure which produces monoclonal antibodies. In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

Antibodies may also be made by recombinant DNA methods. DNA encoding the disclosed antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques.

Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Transgenic animals (e.g., mice) that are capable, upon immunization, of producing a full repertoire of human antibodies in the absence of endogenous immunoglobulin production can be employed. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human antibodies upon antigen challenge.

Optionally, the antibodies are generated in other species and “humanized” for administration in humans Humanized forms of non-human (e.g., murine) antibodies are chimeric immunoglobulins, immunoglobulin chains or fragments thereof (such as Fv, Fab, Fab′, F(ab′).sub.2, or other antigen-binding subsequences of antibodies) which contain minimal sequence derived from non-human immunoglobulin. Humanized antibodies include human immunoglobulins (recipient antibody) in which residues from a complementarity determining region (CDR) of the recipient antibody are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also contain residues that are found neither in the recipient antibody nor in the imported CDR or framework sequences. In general, the humanized antibody will contain substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will contain at least a portion of an immunoglobulin constant region (Fc), typically that of a human immunoglobulin.

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain Antibody humanization techniques generally involve the use of recombinant DNA technology to manipulate the DNA sequence encoding one or more polypeptide chains of an antibody molecule. Humanization can be essentially performed by substituting rodent CDRs or CDR sequences for the corresponding sequences of a human antibody. Accordingly, a humanized form of a non-human antibody (or a fragment thereof) is a chimeric antibody or fragment, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody. Another method uses a particular framework derived from the consensus sequence of all human antibodies of a particular subgroup of light or heavy chains. The same framework may be used for several different humanized antibodies.

Sometimes, CDR-grafting alone can lead to a reduction or complete loss of binding affinity, as a set of supporting framework residues in the Vernier zone are important for maintaining the conformation of the CDRs (Foote and Winter, J. Mol. Bio., 224:487-499 (1992)). This problem can be addressed by reintroducing murine residues into the human framework (Queen, et al., Proc. Natl. Acad. Sci. USA, 86(24):10029-33 (1989)); such substitutions are commonly called back-mutations.

Most therapeutic proteins are, to a varying extent, immunogenic (Van Walle et al., Expert Opin. Biol., Ther., 7:405-418 (2007), Stas et al., Cambridge University Press, Cambridge, (2009)) and even so called fully-human antibody therapeutics may contain immunogenic regions (Harding et al., J. Chromatogr. B. Biomed. Sci. Appl., 752:233-245 (2001)) Immunogenicity is the ability to induce a Th (T-helper) response, which is triggered when a unique T-cell receptor recognizes a peptide bound to the HLA class II molecules displayed on antigen presenting cells. The peptides are generated from proteins internalized by the antigen presenting cell which are then processed through the endosomal cleavage pathway. Only peptides with sufficient affinity for the HLA class II molecules will be presented on the cell surface, and could possibly trigger a Th response.

Consequently, it is possible to lower the immunogenicity potential by removing Th epitopes, a process known as deimmunization (Chamberlain, The Regulatory Review, 5:4-9 (2002), Baker and Jones, Curr. Opin. Drug. Discov. Devel., 10:219-227 (2007)). This is achieved by predicting which peptides in the therapeutic protein can bind to HLA class II molecules, and subsequently introducing substitutions that eliminate or reduce the peptide binding affinity for HLA class II molecules.

There are several HLA class II genes and almost all are highly polymorphic. Additionally, HLA class II molecules consist of an alpha and beta chain, each derived from a different gene which, with the inherent polymorphism, further increases variation. Every individual expresses the genes: DRA/DRB, DQA/DQB and DPA/DPB. Of these only DRA is non-polymorphic. In addition, a ‘second’ DRB gene (DRB3, DRB4 or DRB5) may also be present, the product of which also associates with the DRA chain.

The focus during a deimmunization is on the DR allotypes, which are known to express at a higher level than DQ and DP (Laupeze et al., Hum. Immunol., 61:591-97 (1999), Gansbacher and Zier, Cell Immunol., 117:22-34 (1988), Berdoz, et al., J. Immunol., 139:1336-1341 (1987), Stunz et al., “HLA-DRB 1 abd-DRB4 genes are differentially regulated at the transcriptional level, J. Immunol., 143:3081-3086 (1989)). The assessment of severity for individual epitopes is based on the criteria of promiscuity, i.e., the number of HLA allotypes a specific epitope binds to, as well as the importance (frequency) of the allotypes in the population and a qualitative assessment of the HLA:peptide complex binding strength. As the T-cell population of an individual has been selected to not recognize “self-peptides” it is possible to screen the protein that is being deimmunized for peptides that correspond to (known) self-peptides which should not normally induce a Th response.

Because an important property of a therapeutic antibody is the binding activity, it is important that substitutions proposed during the humanization and deimmunization do not substantially affect the affinity or stability of the antibody. A large amount of information has been collected in the last 20 years on humanization and grafting of the CDRs (Jones et al., Nature, 321, 522-525 (1986), Foote and Winter, J. Mol. Bio., 224:487-499 (1992)), the biophysical properties of antibodies (Ewert et al., J. Mol. Biol., 325:531-553 (2003)), the conformation of the CDR-loops (Chothia and Lesk, J. Mol. Biol., 196:901-917 (1987), Al-Lazikani, et al., J. Mol. Biol., 273:927-948 (1997), North, et al., J. Mol. Biol., 406:228-256 (2011)) and for the frameworks (Vargas-Madrazo and Paz-Garcia, J. Mol. Recognit., 16:113-120 (2003), Honegger, et al., Protein Eng. Des. Sel., 22:121-134 (2009)), which along with advances in protein modeling (Desmet, et al., Proteins, 48:31-43 (2002), Almagro, et al., Proteins, 79:3050-3066 (2011)) makes it possible to accurately humanize and deimmunize antibodies with substantially retained binding affinity and stability.

For example, humanized antibodies can be prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding.

Methods for the production of single-chain antibodies are well known to those of skill in the art. A single chain antibody is created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation. These Fvs lack the constant regions (Fc) present in the heavy and light chains of the native antibody.

In vitro methods are also suitable for preparing monovalent antibodies. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment, called the F(ab′)₂ fragment, that has two antigen combining sites and is still capable of cross-linking antigen.

The Fab fragments produced in the antibody digestion also contain the constant domains of the light chain and the first constant domain of the heavy chain. Fab′ fragments differ from Fab fragments by the addition of a few residues at the carboxy terminus of the heavy chain domain including one or more cysteines from the antibody hinge region. The F(ab′)2 fragment is a bivalent fragment comprising two Fab′ fragments linked by a disulfide bridge at the hinge region. Fab′-SH is the designation herein for Fab′ in which the cysteine residue(s) of the constant domains bear a free thiol group. Antibody fragments originally were produced as pairs of Fab′ fragments which have hinge cysteines between them. Other chemical couplings of antibody fragments are also known.

The antibody can be a hybrid antibody. In hybrid antibodies, one heavy and light chain pair is homologous to that found in an antibody raised against one epitope, while the other heavy and light chain pair is homologous to a pair found in an antibody raised against another epitope. This results in the property of multi-functional valency, i.e., a bivalent antibody has the ability to bind at least two different epitopes simultaneously. Such hybrids can be formed by fusion of hybridomas producing the respective component antibodies, or by recombinant techniques. Such hybrids may, of course, also be formed using chimeric chains.

The targeting function of the antibody can be used therapeutically by coupling the antibody or a fragment thereof with a therapeutic agent. Such coupling of the antibody or fragment (e.g., at least a portion of an immunoglobulin constant region (Fc)) with the therapeutic agent can be achieved by making an immunoconjugate or by making a fusion protein, comprising the antibody or antibody fragment and the therapeutic agent.

Such coupling of the antibody or fragment with the therapeutic agent can be achieved by making an immunoconjugate or by making a fusion protein, or by linking the antibody or fragment to a nucleic acid such as an siRNA, comprising the antibody or antibody fragment and the therapeutic agent.

In some embodiments, the antibody is modified to alter its half-life. In some embodiments, it is desirable to increase the half-life of the antibody so that it is present in the circulation or at the site of treatment for longer periods of time. For example, it may be desirable to maintain titers of the antibody in the circulation or in the location to be treated for extended periods of time. Antibodies can be engineered with Fc variants that extend half-life, e.g., using Xtend™ antibody half-life prolongation technology (Xencor, Monrovia, Calif.).

One method of producing proteins such as antibodies is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the antibody, for example, can be synthesized by standard chemical reactions. For example, a peptide or polypeptide can be synthesized and not cleaved from its synthesis resin whereas the other fragment of an antibody can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. Alternatively, the peptide or polypeptide is independently synthesized in vivo as described above. Once isolated, these independent peptides or polypeptides may be linked to form an antibody or anitgen binding fragment thereof via similar peptide condensation reactions. For example, enzymatic ligation of cloned or synthetic peptide segments allow relatively short peptide fragments to be joined to produce larger peptide fragments, polypeptides or whole protein domains. Alternatively, native chemical ligation of synthetic peptides can be utilized to synthetically construct large peptides or polypeptides from shorter peptide fragments. This method consists of a two-step chemical reaction. The first step is the chemoselective reaction of an unprotected synthetic peptide-alpha-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site.

### Nucleic Acids

In some embodiments, an agent useful in the methods of the invention comprises a nucleic acid molecule. In some embodiments, the nucleic acid molecule is capable of modulating the expression of NEU1 or a component of a NEU1 containing complex. In some embodiments, the nucleic acid molecule comprises a nucleotide sequence that binds to at least a portion of a nucleotide sequence of NEU1. The nucleic acid molecule can be of any length, so long as at least part of the molecule hybridizes sufficiently and specifically to NEU1 mRNA. The nucleic acid molecule can bind to any region of the mRNA. In some embodiments, the nucleotide sequence of NEU1 cDNA is shown in SEQ ID NO: 1 (Genbank Reference Sequences: CR456717, CR541916, and NM_000434.4).

In some embodiments, a region of the nucleic acid molecule is at least 70%, 75%, 80%, 85%, 90%, 91%, 92%, 93%, 94%, 95%, 96%, 97%, 98% or 99% complementary to at least a portion of SEQ ID NO:1.

In some embodiments, the composition can comprise a DNA molecule, such as an antisense DNA molecule. In some embodiments, the composition can comprise an RNA molecule, such as an anti-sense RNA molecule, a small interfering RNA (siRNA) molecule, or small hairpin RNA (shRNA) molecule, which may or may not be comprised on a vector, including a viral vector (such as an adeno-associated viral vector, an adenoviral vector, a retroviral vector, or a lentiviral vector) or a non-viral vector. In some embodiments, the expression of the DNA or RNA molecule may be regulated by a regulatory region present in the cancer cells.

The NEU1-inhibiting agent can be an RNA interference molecule, the RNA interference molecule may be a shRNA, siRNA, miRNA, or guide RNA to CRISPR/CAS9 CRISPRi, etc.

Combinations of shRNAs can also be used in accordance with the present invention.

A target sequence on a target mRNA can be selected from a given cDNA sequence corresponding to NEU1, in some embodiments, beginning 50 to 100 nt downstream (i.e., in the 3′ direction) from the start codon. In some embodiments, the target sequence can, however, be located in the 5′ or 3′ untranslated regions, or in the region nearby the start codon.

In one embodiment, the composition comprises a nucleic acid molecule that comprises a nucleotide sequence that binds to at least a portion of a nucleotide sequence of NEU1 mRNA. In some embodiments, the nucleic acid molecule is a DNA. In some embodiments, the nucleic acid molecule is an RNA.

In some embodiments, the composition comprises an anti-sense DNA. Anti-sense DNA binds with mRNA and prevents translation of the mRNA. The anti-sense DNA can be complementary to a portion of NEU1 mRNA. In some embodiments, the anti-sense DNA is complementary to the entire reading frame. In some embodiments, the anti-sense DNA is complementary to the entire reading frame of SEQ ID NO:1. In some embodiments, the antisense DNA is complementary to a portion of SEQ ID NO:1. In some embodiments, the antisense DNA is at least 15 nucleotides, at least 20 nucleotides, at least 25 nucleotides, at least 30 nucleotides, at least 35 nucleotides, at least 40 nucleotides, at least 50 nucleotides, at least 75 nucleotides, at least 100 nucleotides, at least 150 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, or at least 4500 nucleotides.

In some embodiments, the composition comprises an anti-sense RNA. Anti-sense RNA binds with mRNA and prevents translation of the mRNA. The anti-sense RNA can be complementary to a portion of NEU1 mRNA. In some embodiments, the anti-sense RNA is complementary to the entire reading frame of NEU1. In some embodiments, the anti-sense RNA is complementary to the entire reading frame of SEQ ID NO: 1. In some embodiments, the antisense RNA is complementary to a portion of SEQ ID NO:1. In some embodiments, the antisense RNA is at least 15 nucleotides, at least 20 nucleotides, at least 25 nucleotides, at least 30 nucleotides, at least 35 nucleotides, at least 40 nucleotides, at least 50 nucleotides, at least 75 nucleotides, at least 100 nucleotides, at least 150 nucleotides, at least 200 nucleotides, at least 300 nucleotides, at least 400 nucleotides, at least 500 nucleotides, at least 600 nucleotides, at least 700 nucleotides, at least 800 nucleotides, at least 900 nucleotides, at least 1000 nucleotides, at least 1500 nucleotides, at least 2000 nucleotides, at least 2500 nucleotides, at least 3000 nucleotides, at least 3500 nucleotides, at least 4000 nucleotides, or at least 4500 nucleotides.

In some embodiments, the composition is an siRNA targeting NEU1. SiRNAs are small single or dsRNAs that do not significantly induce the antiviral response common among vertebrate cells but that do induce target mRNA degradation via the RNAi pathway. The term siRNA refers to RNA molecules that have either at least one double stranded region or at least one single stranded region and possess the ability to effect RNA interference (RNAi). It is specifically contemplated that siRNA can refer to RNA molecules that have at least one double stranded region and possess the ability to effect RNAi. The dsRNAs (siRNAs) may be generated by various methods including chemical synthesis, enzymatic synthesis of multiple templates, digestion of long dsRNAs by a nuclease with RNAse III domains, and the like. An “siRNA directed to” at least a particular region of NEU1 means that a particular NEU1 siRNA includes sequences that result in the reduction or elimination of expression of the target gene, i.e., the siRNA is targeted to the region or gene.

The nucleotide sequence of the siRNA is defined by the nucleotide sequence of its target gene. The NEU1 siRNA contains a nucleotide sequence that is essentially identical to at least a portion of the target gene. In some embodiments, the siRNA contains a nucleotide sequence that is completely identical to at least a portion of the NEU1 gene. Of course, when comparing an RNA sequence to a DNA sequence, an “identical” RNA sequence will contain ribonucleotides where the DNA sequence contains deoxyribonucleotides, and further that the RNA sequence will typically contain a uracil at positions where the DNA sequence contains thymidine.

In some embodiments, a NEU1 siRNA comprises a double stranded structure, the sequence of which is “substantially identical” to at least a portion of the target gene. “Identity,” as known in the art, is the relationship between two or more polynucleotide (or polypeptide) sequences, as determined by comparing the sequences. In the art, identity also means the degree of sequence relatedness between polynucleotide sequences, as determined by the match of the order of nucleotides or amino acids between such sequences. Unless otherwise stated, sequence identity/similarity values provided herein refer to the value obtained using the BLAST 2.0 suite of programs using default parameters (Altschul, et al., (1997) Nucleic Acids Res. 25:3389-402).

One of skill in the art will appreciate that two polynucleotides of different lengths may be compared over the entire length of the longer fragment. Alternatively, small regions may be compared. Normally sequences of the same length are compared for a final estimation of their utility in the practice of the present invention. In some embodiments, there is 100% sequence identity between the dsRNA for use as siRNA and at least 15 contiguous nucleotides of the target gene, although a dsRNA having 70%, 75%, 80%, 85%, 90%, or 95% or greater may also be used in the present invention. A siRNA that is essentially identical to a least a portion of the target gene may also be a dsRNA wherein one of the two complementary strands (or, in the case of a self-complementary RNA, one of the two self-complementary portions) is either identical to the sequence of that portion or the target gene or contains one or more insertions, deletions or single point mutations relative to the nucleotide sequence of that portion of the target gene. siRNA technology thus has the property of being able to tolerate sequence variations that might be expected to result from genetic mutation, strain polymorphism, or evolutionary divergence.

In some embodiments, the invention provides an NEU1 siRNA that is capable of triggering RNA interference, a process by which a particular RNA sequence is destroyed (also referred to as gene silencing). In specific embodiments, GPR64 siRNA are dsRNA molecules that are 100 bases or fewer in length (or have 100 base pairs or fewer in its complementarity region). In some embodiments, a dsRNA may be 15, 16, 17, 18, 19, 20, 21, 22, 23, 24, 25, 26, 27, 28, 29, 30, 40, 50, 60, 70, 80, 90, 100 110, 120, 130, 140, 150, 160, 170, 180, 190, 200, 225, 250, 275, 300, 325, 350, 375, 400, 450, 500, 550, 600, 650, 700, 750, 800, 850, 900, 950, or 1000 nucleotides or more in length. In certain embodiments, NEU1 siRNA may be approximately 21 to 25 nucleotides in length. In some cases, it has a two nucleotide 3′ overhang and a 5′ phosphate. The particular NEU1 RNA sequence is targeted as a result of the complementarity between the dsRNA and the particular NEU1 RNA sequence. It will be understood that dsRNA or siRNA of the disclosure can effect at least a 20, 30, 40, 50, 60, 70, 80, 90 percent or more reduction of expression of a targeted NEU1 RNA in target cell. dsRNA of the invention (the term “dsRNA” will be understood to include “siRNA” and/or “candidate siRNA”) is distinct and distinguishable from antisense and ribozyme molecules by virtue of the ability to trigger RNAi. Structurally, dsRNA molecules for RNAi differ from antisense and ribozyme molecules in that dsRNA has at least one region of complementarity within the RNA molecule. In some embodiments, the complementary (also referred to as “complementarity”) region comprises at least or at most 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 contiguous bases. In some embodiments, long dsRNA are employed in which “long” refers to dsRNA that are 1000 bases or longer (or 1000 base pairs or longer in complementarity region). The term “dsRNA” includes “long dsRNA”, “intermediate dsRNA” or “small dsRNA” (lengths of 2 to 100 bases or base pairs in complementarity region) unless otherwise indicated. In some embodiments, dsRNA can exclude the use of siRNA, long dsRNA, and/or “intermediate” dsRNA (lengths of 100 to 1000 bases or base pairs in complementarity region).

It is specifically contemplated that a dsRNA may be a molecule comprising two separate RNA strands in which one strand has at least one region complementary to a region on the other strand. Alternatively, a dsRNA includes a molecule that is single stranded yet has at least one complementarity region as described above (such as when a single strand with a hairpin loop is used as a dsRNA for RNAi). For convenience, lengths of dsRNA may be referred to in terms of bases, which simply refers to the length of a single strand or in terms of base pairs, which refers to the length of the complementarity region. It is specifically contemplated that embodiments discussed herein with respect to a dsRNA comprised of two strands are contemplated for use with respect to a dsRNA comprising a single strand, and vice versa. In a two-stranded dsRNA molecule, the strand that has a sequence that is complementary to the targeted mRNA is referred to as the “antisense strand” and the strand with a sequence identical to the targeted mRNA is referred to as the “sense strand.” Similarly, with a dsRNA comprising only a single strand, it is contemplated that the “antisense region” has the sequence complementary to the targeted mRNA, while the “sense region” has the sequence identical to the targeted mRNA. Furthermore, it will be understood that sense and antisense region, like sense and antisense strands, are complementary (i.e., can specifically hybridize) to each other.

Strands or regions that are complementary may or may not be 100% complementary (“completely or fully complementary”). It is contemplated that sequences that are “complementary” include sequences that are at least 50% complementary, and may be at least 50%, 60%, 70%, 80%, or 90% complementary. In some embodiments, siRNA generated from sequence based on one organism may be used in a different organism to achieve RNAi of the cognate target gene. In other words, siRNA generated from a dsRNA that corresponds to a human gene may be used in a mouse cell if there is the requisite complementarity, as described above. Ultimately, the requisite threshold level of complementarity to achieve RNAi is dictated by functional capability. It is specifically contemplated that there may be mismatches in the complementary strands or regions. Mismatches may number at most or at least 1, 2, 3, 4, 5, 6, 7, 8, 9, 10, 11, 12, 13, 14, 15, 16, 17, 18, 19, 20, 21, 22, 23, 24, 25 residues or more, depending on the length of the complementarity region.

In some embodiments, the single RNA strand or each of two complementary double strands of a dsRNA molecule may be of at least or at most the following lengths: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more (including the full-length NEU1 mRNA without the poly-A tail) bases or base pairs. If the dsRNA is composed of two separate strands, the two strands may be the same length or different lengths. If the dsRNA is a single strand, in addition to the complementarity region, the strand may have 1, 2, 3, 4, 5, 6, 7, 8, 9, 10, 11, 12, 13, 14, 15, 16, 17, 18, 19, 20, 21, 22, 23, 24, 25, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more bases on either or both ends (5′ and/or 3′) or as forming a hairpin loop between the complementarity regions.

In some embodiments, the strand or strands of dsRNA are 100 bases (or base pairs) or less. In specific embodiments the strand or strands of the dsRNA are less than 70 bases in length. With respect to those embodiments, the dsRNA strand or strands may be from 5-70, 10-65, 20-60, 30-55, 40-50 bases or base pairs in length. A dsRNA that has a complementarity region equal to or less than 30 base pairs (such as a single stranded hairpin RNA in which the stem or complementary portion is less than or equal to 30 base pairs) or one in which the strands are 30 bases or fewer in length is specifically contemplated, as such molecules evade a mammalian's cell antiviral response. Thus, a hairpin dsRNA (one strand) may be 70 or fewer bases in length with a complementary region of 30 base pairs or fewer. In some cases, a dsRNA may be processed in the cell into siRNA.

The siRNA of the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion.

One or both strands of the siRNA of the disclosure can comprise a 3′ overhang. As used herein, a “3′ overhang” refers to at least one unpaired nucleotide extending from the 3′-end of a duplexed RNA strand.

Thus in some embodiments, the NEU1 siRNA of the invention comprises at least one 3′ overhang of from 1 to about 6 nucleotides (which includes ribonucleotides or deoxynucleotides) in length, from 1 to about 5 nucleotides in length, from 1 to about 4 nucleotides in length, or from about 2 to about 4 nucleotides in length.

In some embodiments in which both strands of the NEU1 siRNA molecule comprise a 3′ overhang, the length of the overhangs can be the same or different for each strand. In some embodiments, the 3′ overhang is present on both strands of the siRNA, and is 2 nucleotides in length. For example, each strand of the NEU1 siRNA of the invention can comprise 3′ overhangs of dithymidylic acid (“TT”) or diuridylic acid (“UU”).

In order to enhance the stability of the present NEU1 siRNA, the 3′ overhangs can be also stabilized against degradation. In some embodiments, the overhangs are stabilized by including purine nucleotides, such as adenosine or guanosine nucleotides. In some embodiments, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine can significantly enhance the nuclease resistance of the 3′ overhang in tissue culture medium.

In some embodiments, the NEU1 siRNA of the disclosure can be targeted to any stretch of approximately 19-25 contiguous nucleotides in any of the target mRNA sequences (the “target sequence”). Techniques for selecting target sequences for siRNA are given, for example, in Tuschl T et al., “The siRNA User Guide,” revised Oct. 11, 2002, the entire disclosure of which is herein incorporated by reference. “The siRNA User Guide” is available on the world wide web at a website maintained by Dr. Thomas Tuschl, Department of Cellular Biochemistry, AG 105, Max-Planck-Institute for Biophysical Chemistry, 37077 Gottingen, Germany, and can be found by accessing the website of the Max Planck Institute and searching with the keyword “siRNA.” Thus, in some embodiments, the sense strand of the present siRNA comprises a nucleotide sequence identical to any contiguous stretch of about 19 to about 25 nucleotides in the target mRNA.

In some embodiments, the siRNA comprises a 21 nucleotide double stranded sequence. In some embodiments, the siRNA comprises a two-TT overhang (Yang et al., Nucleic Acid Research, 34(4), 1224-1236, 2006).

In some embodiments, the composition useful in the methods of the invention comprises an shRNA molecule that targets NEU1 mRNA (NEU1 shRNA). shRNA is an artificial RNA molecule with a tight hairpin turn that can be used to silence target gene expression via RNA interference (RNAi). In certain cases, expression of NEU1 shRNA in cells is achieved through delivery of non-viral vectors (such as plasmids or bacterial vectors) or through viral vectors. shRNA is useful because it has a relatively low rate of degradation and turnover.

In order to obtain long-term gene silencing, expression vectors that continually express siRNAs in stably transfected mammalian cells can be used (Brummelkamp et al., Science 296: 550-553, 2002; Lee et al., Nature Biotechnol. 20:500-505, 2002; Miyagishi, M, and Taira, K. Nature Biotechnol. 20:497-500, 2002; Paddison, et al., Genes & Dev. 16:948-958, 2002; Paul et al., Nature Biotechnol. 20:505-508, 2002; Sui, Proc. Natl. Acad. Sci. USA 99(6):5515-5520, et al., 2002; Yu et al., Proc. Natl. Acad. Sci. USA 99(9):6047-6052, 2002). Many of these plasmids have been engineered to express shRNAs lacking poly (A) tails. Transcription of shRNAs is initiated at a polymerase III (pol III) promoter and is believed to be terminated at position 2 of a 4-5-thymine transcription termination site. Upon expression, shRNAs are thought to fold into a stem-loop structure with 3′ UU-overhangs. Subsequently, the ends of these shRNAs are processed, converting the shRNAs into ^{˜}21 nt siRNA-like molecules. The siRNA-like molecules can, in turn, bring about gene-specific silencing in the transfected mammalian cells.

The length of the stem and loop of shRNAs can be varied. In some embodiments, stem lengths could range anywhere from 25 to 29 nucleotides and loop size could range between 4 to 23 nucleotides without affecting silencing activity. Moreover, presence of G-U mismatches between the two strands of the shRNA stem does not necessarily lead to a decrease in potency.

In some embodiments, the present invention is directed to methods of administering subjects with compositions comprising expression vectors and/or chemically synthesized shRNA molecules that target NEU1. In some embodiments, the composition comprises a nucleotide sequence expressing a small hairpin RNA (shRNA) molecule. In some embodiments, the expression vector is a lentivirus expression vector.

In some embodiments, it is contemplated that nucleic acids or other active agents of the invention may be labeled. The label may be fluorescent, radioactive, enzymatic, or calorimetric. It is contemplated that a dsRNA may have one label attached to it or it may have more than one label attached to it. When more than one label is attached to a dsRNA, the labels may be the same or be different. If the labels are different, they may appear as different colors when visualized. The label may be on at least one end and/or it may be internal. Furthermore, there may be a label on each end of a single stranded molecule or on each end of a dsRNA made of two separate strands. The end may be the 3′ and/or the 5′ end of the nucleic acid. A label may be on the sense strand or the sense end of a single strand (end that is closer to sense region as opposed to antisense region), or it may be on the antisense strand or antisense end of a single strand (end that is closer to antisense region as opposed to sense region). In some cases, a strand is labeled on a particular nucleotide (G, A, U, or C). When two or more differentially colored labels are employed, fluorescent resonance energy transfer (FRET) techniques may be employed to characterize the dsRNA.

Labels contemplated for use in several embodiments are non-radioactive. In many embodiments of the invention, the labels are fluorescent, though they may be enzymatic, radioactive, or positron emitters. Fluorescent labels that may be used include, but are not limited to, BODIPY, Alexa Fluor, fluorescein, Oregon Green, tetramethylrhodamine, Texas Red, rhodamine, cyanine dye, or derivatives thereof. The labels may also more specifically be Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5, DAPI, 6-FAM, Killer Red, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, SYPRO, TAMRA, TET, Tetramethylrhodamine, and/or Texas Red. A labeling reagent is a composition that comprises a label and that can be incubated with the nucleic acid to effect labeling of the nucleic acid under appropriate conditions. In some embodiments, the labeling reagent comprises an alkylating agent and a dye, such as a fluorescent dye. In some embodiments, a labeling reagent comprises an alkylating agent and a fluorescent dye such as Cy3, Cy5, or fluorescein (FAM). In still further embodiments, the labeling reagent is also incubated with a labeling buffer, which may be any buffer compatible with physiological function (i.e., buffers that is not toxic or harmful to a cell or cell component) (termed “physiological buffer”).

In some embodiments, the nucleic acids of the invention can be modified. In some embodiments, the nucleic acids can be modified to include a phosphorothioate (PS) backbone. The modification to the backbone can be throughout the molecule or at one or more defined sites. In some embodiments, the nucleic acids can be modified to encompass peptide nucleic acids (PNA). In some embodiments, the nucleic acids can be modified to encompass phosphorodiamidate morpholino oligomers (PMO).

In some embodiments, the nucleic acid molecules of the invention can include derivatives such as S-oligonucleotides (phosphorothioate derivatives or S-oligos). S-oligos (nucleoside phosphorothioates) are isoelectronic analogs of an oligonucleotide (O-oligo) in which a nonbridging oxygen atom of the phosphate group is replaced by a sulfur atom. The S-oligos of the present invention may be prepared by treatment of the corresponding 0-oligos with 3H-1,2-benzodithiol-3-one-1,1-dioxide which is a sulfur transfer reagent. See Iyer et al., J. Org. Chem. 55:4693-4698 (1990); and Iyer et al., J. Am. Chem. Soc. 112:1253-1254 (1990), the disclosures of which are fully incorporated by reference herein.

In some embodiments of the invention, a dsRNA has one or more non-natural nucleotides, such as a modified residue or a derivative or analog of a natural nucleotide. Any modified residue, derivative or analog may be used to the extent that it does not eliminate or substantially reduce (by at least 50%) RNAi activity of the dsRNA.

A person of ordinary skill in the art is well aware of achieving hybridization of complementary regions or molecules. Such methods typically involve heat and slow cooling of temperature during incubation, for example.

In some embodiments, the nucleic acid molecules of the present methods are encoded by expression vectors. The expression vectors may be obtained and introduced into a cell. Once introduced into the cell the expression vector is transcribed to produce various nucleic acids. Expression vectors include nucleic acids that provide for the transcription of a particular nucleic acid. Expression vectors include plasmid DNA, linear expression elements, circular expression elements, viral expression constructs (including adenoviral, adeno-associated viral, retroviral, lentiviral, and so forth), and the like, all of which are contemplated as being used in the compositions and methods of the present disclosure. In some embodiments one or at least 2, 3, 4, 5, 6, 7, 8, 9, 10 or more nucleic acid molecules binding to NEU1 RNA are encoded by a single expression construct. Expression of the nucleic acid molecules binding to NEU1 RNA may be independently controlled by at least 1, 2, 3, 4, 5, 6, 7, 8, 9, 10 or more regulatory elements. In certain embodiments, at least 1, 2, 3, 4, 5, 6, 7, 8, 9, 10 or more expression constructs can be introduced into a cell. Each expression construct can encode 1, 2, 3, 4, 5, 6, 7, 8, 9, 10 or more nucleic acid molecules binding to NEU1 RNA. In some embodiments, nucleic acid molecules binding to NEU1 RNA may be encoded as expression domains. Expression domains include a transcription control element, which may or may not be independent of other control or promoter elements; a nucleic acid; and optionally a transcriptional termination element.

In some embodiments, the invention provides nucleic acid molecules encoding dominant negative NEU1, which can include dominantly negative active fragments or derivatives of the wild type sequences. In some embodiments, the nucleic acid molecule is packaged in a viral vector. In some embodiments, the dominant negative NEU1 or biologically active fragments or derivatives thereof encodes a protein that is at least 90% identical to SEQ ID NO:1. In some embodiments, the NEU1 or a biologically active fragment or derivative thereof may be derived from genomic DNA, i.e., cloned directly from the genome of a particular organism. In some embodiments, however, the vector comprising NEU1 comprises complementary DNA (cDNA).

The organismal source of NEU1 is not limiting. In some embodiments, the NEU1 nucleic acid sequence is derived from a mammal, bird, reptile or fish. In some embodiments, the NEU1 is of human origin. In some embodiments, the NEU1 is from dog, cat, horse, mouse, rat, guinea pig, sheep, cow, pig, monkey, or ape. The nucleic acid molecules may be produced using recombinant DNA technology (e.g., polymerase chain reaction (PCR) amplification, cloning) or chemical synthesis. NEU1 nucleic acids include natural nucleic acid molecules and homologues thereof, including, but not limited to, natural allelic variants and modified nucleic acid molecules in which nucleotides have been inserted, deleted, substituted, and/or inverted in such a manner that such modifications provide the desired effect.

In some embodiments, the nucleic acid sequence encoding dominant negative NEU1 is at least 60%, 70%, 75%, 80%, 85%, 90%, 91%, 92%, 93%, 94%, 95%, 96%, 97%, 98%, or 99% identical to the coding sequence of SEQ ID NO:1.

Any suitable viral vector can be used in the methods of the invention. For example, vectors derived from adenovirus (AV); adeno-associated virus (AAV; including AAV serotypes); retroviruses (e.g, lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like.

Selection of recombinant viral vectors suitable for use in the invention, are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; and Anderson W F (1998), Nature 392: 25-30, the entire disclosures of which are herein incorporated by reference.

The ability of a RNA of the claimed invention to cause RNAi-mediated degradation of the target mRNA can be evaluated using standard techniques for measuring the levels of RNA or protein in cells. For example, NEU1 siRNA of the invention can be delivered to cultured cells, and the levels of target mRNA can be measured by Northern blot or dot blotting techniques, or by quantitative RT-PCR. Alternatively, the levels of NEU1 protein in the cultured cells can be measured by ELISA or Western blot. A suitable cell culture system for measuring the effect of the present siRNA on target mRNA or protein levels may be utilized. RNAi-mediated degradation of NEU1 mRNA by an siRNA containing a given target sequence can also be evaluated with animal models, for example.

In some embodiments, the nucleic acids can be administered to the subject either as naked nucleic acid, in conjunction with a delivery reagent, or as a recombinant plasmid or viral vector that expresses the nucleic acids. Delivery of nucleic acids or vectors to an individual may occur by any suitable means, but in specific embodiments it occurs by one of the following: cyclodextrin delivery system; ionizable lipids; DPC conjugates; GalNAc-conjugates; self-assembly of oligonucleotide nanoparticles (DNA tetrahedra carrying multiple siRNAs); or polymeric nanoparticles made of low-molecular-weight polyamines and lipids (see Kanasty et al. Nature Materials 12, 967-977 (2013) for review of same).

Suitable delivery reagents for administration in conjunction with the present nucleic acids or vectors include at least the Mirus Transit TKO lipophilic reagent; lipofectin; lipofectamine; cellfectin; or polycations (e.g., polylysine), or liposomes. In specific embodiments, a particular delivery reagent comprises a liposome.

Liposomes can aid in the delivery of the present nucleic acids or vectors to a particular tissue, and can also increase the blood half-life of the nucleic acids. Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example as described in Szoka et al. (1980), Ann. Rev. Biophys. Bioeng. 9: 467; and U.S. Pat. Nos. 4,235,871, 4,501,728, 4,837,028, and 5,019,369, the entire disclosures of which are herein incorporated by reference.

In certain aspects, the liposomes encapsulating the present nucleic acids comprise a ligand molecule that can target the liposome to a particular cell or tissue at or near the site of interest. Ligands that bind to receptors prevalent in the tissues to be targeted, such as monoclonal antibodies that bind to surface antigens, are contemplated. In particular cases, the liposomes are modified so as to avoid clearance by the mononuclear macrophage and reticuloendothelial systems, for example by having opsonization-inhibition moieties bound to the surface of the structure. In one embodiment, a liposome of the invention can comprise both opsonization-inhibition moieties and a ligand. Opsonization-inhibiting moieties for use in preparing the liposomes of the disclosure are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”); e.g., as described in U.S. Pat. No. 4,920,016, the entire disclosure of which is herein incorporated by reference. Liposomes modified with opsonization-inhibition moieties thus remain in the circulation much longer than unmodified liposomes. For this reason, such liposomes are sometimes called “stealth” liposomes.

Stealth liposomes are known to accumulate in tissues fed by porous or “leaky” microvasculature. Thus, target tissue characterized by such microvasculature defects, for example solid tumors, will efficiently accumulate these liposomes; see Gabizon, et al. (1988), P.N.A.S., USA, 18: 6949-53. In addition, the reduced uptake by the RES lowers the toxicity of stealth liposomes by preventing significant accumulation in the liver and spleen. Thus, liposomes of the invention that are modified with opsonization-inhibition moieties can deliver the present nucleic acids to tumor cells.

In some embodiments, opsonization inhibiting moieties suitable for modifying liposomes are water-soluble polymers with a number-average molecular weight from about 500 to about 40,000 Daltons, and in some embodiments. from about 2,000 to about 20,000 Daltons. Such polymers can include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GME Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups.

In some embodiments the opsonization-inhibiting moiety is a PEG, PPG, or derivatives thereof. Liposomes modified with PEG or PEG-derivatives are sometimes called “PEGylated liposomes.” The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60 degrees C.

Recombinant plasmids that express nucleic acids of the invention are discussed above. Such recombinant plasmids can also be administered directly or in conjunction with a suitable delivery reagent, including the Mirus Transit LT 1 lipophilic reagent; lipofectin; lipofectamine; cellfectin; polycations (e.g., polylysine) or liposomes.

The nucleic acids that inhibit the activity of NEU1 can be administered to the subject by any suitable means. For example, the nucleic acids can be administered by gene gun, electroporation, or by other suitable parenteral or enteral administration routes, or by injection, for example, by intramuscular or intravenous injection.

Suitable parenteral administration routes include intravascular administration (e.g. intravenous bolus injection, intravenous infusion, intra-arterial bolus injection, intra-arterial infusion and catheter instillation into the vasculature); peri- and intra-tissue administration (e.g., peri-tumoral and intra-tumoral injection, intra-retinal injection or subretinal injection); subcutaneous injection or deposition including subcutaneous infusion (such as by osmotic pumps); direct (e.g., topical) application to the area at or near the site of interest, for example by a catheter or other placement device (e.g., a corneal pellet or a suppository, eye-dropper, or an implant comprising a porous, non-porous, or gelatinous material); and inhalation. In a particular embodiment, injections or infusions of the composition(s) are given at or near the site of disease.

The nucleic acids that inhibit the activity of NEU1 can be administered in a single dose or in multiple doses. Where the administration of a composition is by infusion, the infusion can be a single sustained dose or can be delivered by multiple infusions. Injection of the agent directly into the tissue is at or near the site of need. Multiple injections of the agent into the tissue at or near the site of interest are encompassed within this disclosure.

One skilled in the art can also readily determine an appropriate dosage regimen for administering the nucleic acids that inhibit the activity of NEU1 to a given subject. For example, the composition(s) can be administered to the subject once, such as by a single injection or deposition at or near the site of interest. In some embodiments, the composition(s) can be administered to a subject once or twice daily to a subject once weekly for a period of from about three to about twenty-eight days, in some embodiments, from about seven to about ten weeks. In some dosage regimens, the composition(s) is injected at or near the site of interest once a day for seven days. Where a dosage regimen comprises multiple administrations, it is understood that the effective amount of composition(s) administered to the subject can comprise the total amount of composition(s) administered over the entire dosage regimen.

### Combination Therapies

In some embodiments, the method further comprises one or more additional treatments. Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s).

In some embodiments, the combination of an agent described herein and at least one additional therapeutic agent results in additive or synergistic results. In some embodiments, the combination therapy results in an increase in the therapeutic index of the agent. In some embodiments, the combination therapy results in an increase in the therapeutic index of the additional therapeutic agent(s). In some embodiments, the combination therapy results in a decrease in the toxicity and/or side effects of the agent. In some embodiments, the combination therapy results in a decrease in the toxicity and/or side effects of the additional therapeutic agent(s).

In certain embodiments, in addition to administering a composition that inhibits the activity of NEU1 described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second). For example, one or more NEU1 inhibitors can be administered on the same day, or a different day than the second active agent. In some embodiments, the second active agent can be administered on the first, second, third, or fourth day, following or before one or more inhibitors of NEU1.

In some embodiments, the additional therapeutic agent is Pirfenidone. Pirfenidone (ESBRIET, PIRESPA, ETUARY) is an anti-scarring (anti-fibrotic) medication that slows the progression of IPF. Some patients taking pirfenidone have side effects, most commonly stomach upset and skin rash, particularly with exposure to sun.

In some embodiments, the additional therapeutic agent is Nintedanib (VARGATEF, OFEV). Nintedanib is an anti-scarring (anti-fibrotic) medication that slows progression of IPF. Some patients taking nintedanib have side effects, most commonly including diarrhea.

In some embodiments, the additional therapeutic agent is a corticosteroid, (for example, prednisone oral pills, ORASONE, or ADASONE), which can reduce inflammation in lungs by suppressing the immune system. Corticosteroids are only used in patients with IPF who have an acute exacerbation of their lung fibrosis, and can be harmful in patients with IPF that have scarring that is stable or slowly worsening.

In some embodiments, the additional therapeutic agent is N-Acetylcysteine (NAC; oral or aerosolized; MUCOMYST). NAC is an antioxidant that has frequently been used in patients with IPF. A large clinical trial published in May 2014 showed that NAC does not slow progression of IPF.

In some embodiments, the additional therapeutic agent is azathioprine, or cyclophosphamide

In some embodiments, the additional therapeutic agent is oxygen. Some people who have pulmonary fibrosis eventually require continuous oxygen therapy.

Additional classes of drugs that can be combined with one or more inhibitors of NEU1 include anti-neointima agents, chemotherapeutic agents, antibiotics, antivirals, steroidal and non-steroidal anti-inflammatories, conventional immunotherapeutic agents, immune-suppressants, cytokines, chemokines and/or growth factors, anti-proliferatives or anti-migration agents designed for treating or preventing pulmonary fibrosis, agents which affect migration and extracellular matrix production, agents which affect platelet deposition or formation of thrombus, and agents that promote vascular healing and re-endothelialization.

Exemplary antiproliferative agents include, but are not limited to, Paclitaxel (Taxol), QP-2 Vincristin, Methotrexat, Angiopeptin, Mitomycin, BCP 678, Antisense c-myc, ABT 578, Actinomycin-D, RestenASE, 1-Chlor-deoxyadenosin, PCNA Ribozym, and Celecoxib.

Exemplary agents modulating cell replication/proliferation include targets of rapamycin (TOR) inhibitors (including sirolimus, everolimus and ABT-578), paclitaxel and antineoplastic agents, including alkylating agents (e.g., cyclophosphamide, mechlorethamine, chlorambucil, melphalan, carmustine, lomustine, ifosfamide, procarbazine, dacarbazine, temozolomide, altretamine, cisplatin, carboplatin and oxaliplatin), antitumor antibiotics (e.g., bleomycin, actinomycin D, mithramycin, mitomycin C, etoposide, teniposide, amsacrine, topotecan, irinotecan, doxorubicin, daunorubicin, idarubicin, epirubicin, mitoxantrone and mitoxantrone), antimetabolites (e.g., deoxycoformycin, 6-mercaptopurine, 6-thioguanine, azathioprine, 2-chlorodeoxyadenosine, hydroxyurea, methotrexate, 5-fluorouracil, capecitabine, cytosine arabinoside, azacytidine, gemcitabine, fludarabine phosphate and aspariginase), antimitotic agents (e.g., vincristine, vinblastine, vinorelbine, docetaxel, estramustine) and molecularly targeted agents (e.g., imatinib, tretinoin, bexarotene, bevacizumab, gemtuzumab ogomicin and denileukin diftitox).

The additional therapeutic agents can be administered locally or systemically to the subject, or coated or incorporated onto, or into a device or graft. The additional therapeutic reagents can be administered by the same, or by different routes and by different means. For example, one or more NEU1 inhibitors can be delivered via infusion with one or more of paclitaxel, taxotere and other taxoid compounds, methotrexate, anthracyclines such as doxorubicin, everolimus, serolimus, rapamycin or rapamycin derivatives delivered by different means.

Combined administration can include co-administration, either in a single pharmaceutical formulation or using separate formulations, or consecutive administration in either order but generally within a time period such that all active agents can exert their biological activities simultaneously.

It will be appreciated that the combination of an agent that inhibits the activity of NEU1 described herein and at least one additional therapeutic agent may be administered in any order or concurrently. In some embodiments, the agent will be administered to patients that have previously undergone treatment with a second therapeutic agent. In certain other embodiments, the agent that inhibits the activity of NEU1 and a second therapeutic agent will be administered substantially simultaneously or concurrently. For example, a subject may be given an agent while undergoing a course of treatment with a second therapeutic agent. In certain embodiments, a composition that inhibits the activity of NEU1 will be administered within 1 year of the treatment with a second therapeutic agent. In certain alternative embodiments, an agent that inhibits the activity of NEU1 will be administered within 10, 8, 6, 4, or 2 months of any treatment with a second therapeutic agent. In certain other embodiments, an agent that inhibits the activity of NEU1 will be administered within 4, 3, 2, or 1 weeks of any treatment with a second therapeutic agent. In some embodiments, an agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

### Therapeutic Compositions

In another embodiment, the invention provides a composition for treating a fibrotic lung disease or condition in a subject an effective amount of an agent that inhibits the activity of NEU1 sialidase and a pharmaceutically acceptable carrier.

Formulations can be prepared for storage and use by combining the compositions or active agents of the present invention with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy. 22^{st} Edition, 2012, Pharmaceutical Press, London.).

The pharmaceutical compositions of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The agent that inhibits the activity of NEU1 described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22^{st} Edition, 2012, Pharmaceutical Press, London.

In certain embodiments, pharmaceutical formulations include an agent of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In some embodiments, the agent that inhibits the activity of NEU1 can be formulated in a lipid nanoparticle.

In certain embodiments, sustained-release preparations comprising the agent that inhibits the activity of NEU1 described herein can be produced. Suitable examples of sustained-release preparations include semi-permeable matrices of solid hydrophobic polymers containing an agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT (injectable microspheres composed of lactic acid-glycolic acid copolymer and leuprolide acetate), sucrose acetate isobutyrate, and poly-D-(−)-3-hydroxybutyric acid.

While the invention has been described with reference to certain particular examples and embodiments herein, those skilled in the art will appreciate that various examples and embodiments can be combined for the purpose of complying with all relevant patent laws (e.g., methods described in specific examples can be used to describe particular aspects of the invention and its operation even though such are not explicitly set forth in reference thereto).

The present invention is further illustrated by the following Examples. These Examples are provided to aid in the understanding of the invention and are not to be construed as a limitation thereof.

### EXAMPLES

### Example 1—NEU1 Sialidase Regulates T Lymphocyte Infiltration and Fibrosis in the Lung Tissues of Bleomycin-Challenged Mice

We previously demonstrated that expression of the NEU1 sialidase is elevated in the lung tissues of patients with Idiopathic Pulmonary Fibrosis (IPF) (Am J. Physiol. Lung. Cell. Mol. Physiol. 310; L940-L954, 2016). Further, forced NEU1 overexpression in human airway epithelia restrained their migration in wounding assays, in lung microvascular endothelia, increased adhesiveness for lymphocytes and disrupted capillary-like tube formation, and in lung fibroblasts, increased collagen deposition. Since NEU1 expression was elevated in lung tissues of IPF patients, and forced NEU1 overexpression in human lung cells in vitro provoked phenotypic changes compatible with IPF pathogenesis, we asked whether NEU1 catalytic activity might provide a target for therapeutic intervention. As described below in this Example, C57BL/6 mice were intratracheally administered bleomycin. On days 7-14, the mice were intraperitoneally injected daily with equivalent doses of the broad-spectrum sialidase inhibitor, 2-deoxy-N-acetyneuraminic acid (2DN), either of 2 NEU1-selective sialidase inhibitors, C9-BADANA or III-32B5, or vehicle alone. On day 14, mice were sacrificed and bronchoalveolar lavage fluid (BALF) and lungs harvested. The BALF was processed for cell counts while the lung tissues were processed for total sialidase activity for the 4-MU-NANA substrate, qRT-PCR for collagen mRNA, and hydroxyproline-based QuickZyme assays for collagen protein. The bleomycin challenge increased T lymphocytes in the BALF and lymphocyte infiltration and collagen mRNA and protein levels in the lung. Prior sialidase inhibition with 2DN, C9-BADANA, and III-32B5, each protected against lymphocyte recruitment to the lung and collagen expression in the lung, at both mRNA and protein levels, compared to that seen in vehicle-infused controls. These combined data indicate that NEU1-selective inhibition provides a therapeutic intervention for bleomycin-induced pulmonary fibrosis, and, more generally, for profibrotic disease states.

Materials and Methods:

Experimental Animal Models:

Wild-type female C57BL/6 mice aged 10-12 weeks and weighing 18-20 g (The Jackson Laboratory, Bar Harbor, Me.) were treated in accordance with a research protocol approved by the University of Maryland Institutional Animal Care and Use Committee. Animals were maintained in sterile microisolator cages with sterile rodent feed and water in the Baltimore VA Medical Center Research Animal Facility approved by the Association for Assessment and Accreditation of Laboratory Animal Care. To model pulmonary inflammation and fibrosis, a single dose of 0.075 U of bleomycin (BLM)(Sigma-Aldrich, St. Louis, Mo.) diluted in 50 μl of sterile phosphate-buffered saline (PBS) was intratracheally (i.t.) delivered to mouse lungs on day 0. Briefly, a minor anterior midline neck incision was made to reveal the trachea, and a MicroSprayer® (Penn-Century, Wyndmoor, Philadelphia, Pa.) was inserted i.t., and the bleomycin or sterile PBS alone instilled. Mice were serially weighed, and on days 8-14, daily administered intraperitoneally (i. p.) with 15 mg/kg of 2DN, or C9-BADANA, or III-32B5, or vehicle alone. On day 14, mice were euthanized by CO₂ asphyxiation followed by cervical dislocation. Immediately postmortem, bronchoalveolar lavage fluid (BALF)s and lungs were harvested. BALF samples were collected and analyzed. Installation and withdrawal of 1 ml of PBS twice via an 18-gauge blunt-end needle secured in the trachea were performed in each animal The 2 aliquots of BALF were pooled, centrifuged, and total and differential cell counts performed. The lungs were processed for qRT-PCR, to measure levels for NEU 1-4, PPCA, collagen 1α2, and collagen 3α1transcripts, quantitative immunoblotting for NEU1-4 and PPCA proteins, and determination of hydroxyproline as a measure of total collagen. In selected experiments, lung tissue was processed for assays of total sialidase activity for fluorogenic substrate, 2′-(4-methylumbelliferyl)-α-D-N-acetylneuraminic acid (4-MU-NANA) and peanut agglutinin (PNA) lectin blotting as an indicator of desialylation.

Human Lung Cell Cultures: Human primary small airway epithelial cell (SAEC)s isolated from the distal portion of the bronchioles and human primary pulmonary microvascular endothelial cell (HPMEC)s were purchased from Promocell (Heidelberg, Germany) SAECs were cultured in predefined small airway growth medium containing hydrocortisone, human epidermal growth factor, epinephrine, transferrin, insulin, retinoic acid, triiodothyronine, and fatty acid-free bovine serum albumin. Only SAEC passaged 2-4 were studied. HPMECs were cultured in endothelial cell growth medium (MV-2, PromoCell) containing growth medium supplement mix (PromoCell). Only HPMEC passages 4-7 were studied. Primary normal human lung fibroblast (NHLF)s were either isolated from normal lungs initially harvested from lung transportation but ultimately not used in a study approved by the Institutional Review Board at the University of Maryland, or purchased from Lonza (Walkersville, Md.). The NHLFs were cultured in DMEM supplemented with 2.0 mM glutamine, 2.9 mM sodium pyruvate, 50 mg/L gentamicin, and 10% fetal bovine serum. The NHLF cultures were passaged by tyrosination and only passages 3-6 were studied.

Fluorometric Assay for Sialidase Activity: Lungs harvested from mice pretreated with one of three sialidase inhibitors or saline alone were weighed, homogenized, centrifuged, and the supernatants collected. These samples and SAECs, HPMECs, and NHLFs preincubated with increasing concentrations of the NEU1-selective NEU inhibitor, III-32B5 were suspended in 200μλ of 500 mM sodium acetate, pH 4.4 containing 0.1% Triton X-100, and a protease inhibitor mixture (Roche Applied Science), and then incubated for 1 h at 37° C. with 25 μl of 2.0 mM 2′-(4-methylumbelliferyl)-α-D-N-acetylneuraminic acid (4-MU-NANA), mixing the tubes every 15 min. The NEU reaction was terminated by addition of 133 mM glycine, pH 10.3, 60 mM NaCl, and 0,083 M Na₂CO₂ after which the fluorescence intensity was measured with a Bio-Rad fluorometer (excitation at 355 nm; emission at 460 nm).

qRT-PCR for collagens 1α2 and 3α1, NEU1-4, and PPCA: Total cellular RNA was extracted from murine lung tissue. RNA purity was established with the A₂₆₀/A₂₈₀ absorption ratio (>1.90). Total RNA (1.0 μg) was treated with DNase I (Invitrogen) for 15 min and reverse transcribed using avian myeloblastosis virus reverse transcriptase and poly(T) primer (Promega). The resulting cDNA was quantified by real time quantitative reverse transcription-polymerase chain reaction (qRT-PCR) using SYBR Green PCR Master Mix and ABI Prism 7900HT cycler. Primers for murine collagens 1α2 and 3α1, NEU1-4, and PPCA mRNAs were designed using Primer Express 2.0 (Applied Biosystems, Foster City, Calif.). Relative collagen 1α2 and 3α1, NEU1-4, and PPCA gene expression was calculated using the 2-ΔCT method where collagen 1α2 and 3α1, NEU1-4, and PPCA transcripts were normalized to the levels of 18S rRNA transcripts as the internal control.

Immunoblotting for NEU1-4, and PPCA:

Murine lung tissues were thoroughly rinsed with ice-cold HEPES buffer and lysed with ice-cold 50 mM Tris-HCl, pH 8.0, 1.0% Nonidet P-40, 0.5% sodium dodecyl sulfate (SDS), 150 mM NaCl, 0.1 mM phenylmethylsulphonyl fluoride, 5.0 μg/ml leupeptin, 1.0 mg/ml pepstatin A, 1.0 mg/ml aprotinin, 1.0 mM vanadate, 1.0 mM sodium fluoride, 10 mM disodium pyrophosphate, 500 μM p-nitrophenol, and 1.0 mM phenylarsine oxide. The lung samples assayed for protein concentration with Bio-Rad Protein Assay Dye Reagent. Equal amounts of protein were resolved by electrophoresis on 8-16% SDS-polyacrylamide gels and transferred to polyvinylidene difluoride (PVDF) membranes. In some experiments, the blots were blocked for 1 h using 5.0% nonfat milk in 50 mM Tris-HCl, pH 8.0, 150 mM NaCl, and 0.01% Tween 20 (TB S-T), probed with rabbit anti-human NEU 1-4 antibodies (OriGene, Rockville, Md.), rabbit anti-human cathepsin A antibody (Abcam, Cambridge, Mass.), each followed HRP goat anti-rabbit antibody (Cell Signaling, Danvers, Mass.), and developed with enhanced chemiluminescence (ECL) reagents, To confirm equivalent protein loading and transfer, blots were stripped with Re-Blot Plus Strong Solution (Millipore, Burlington, Mass.), washed with TBS-T, reprobed with rabbit GAPDH antibody followed by HRP-conjugated goat anti-rabbit antibody, and developed with ECL reagents.

Measurement of Total Lung Collagen: Collagen content was measured in lung tissue based on detection of hydroxyproline, using the Total Collagen Assay kit from QuickZyme (Leiden, The Netherlands). Briefly, following hydrolysis of 50 mg lung tissue in 500 ml of 6M HCl for 20 h at 95° C., the hydrolysate was diluted 10-fold with 4M HCl and measured against serial dilutions of collagen standard; the results were expressed as μg collagen per mg wet lung tissue.

### Results:

Pharmacologic Blockade of Total Sialidase Catalytic Activity in Mouse Lung In Vivo:

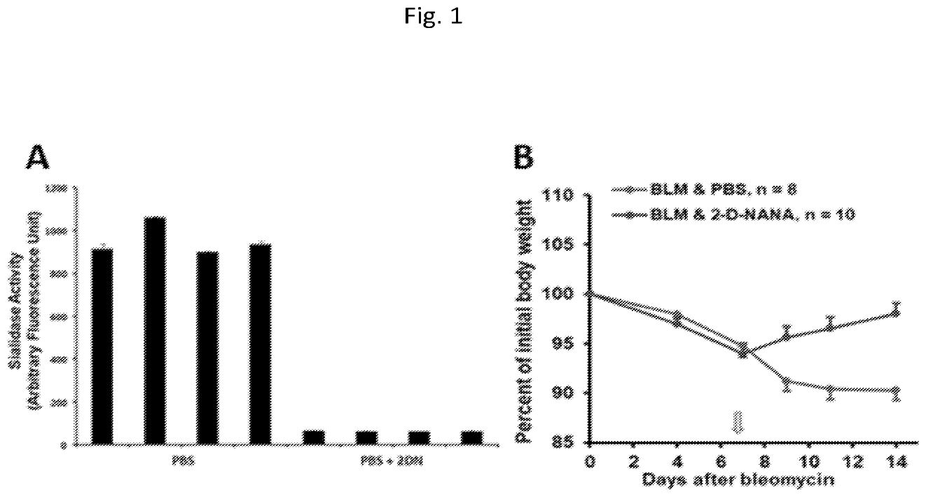

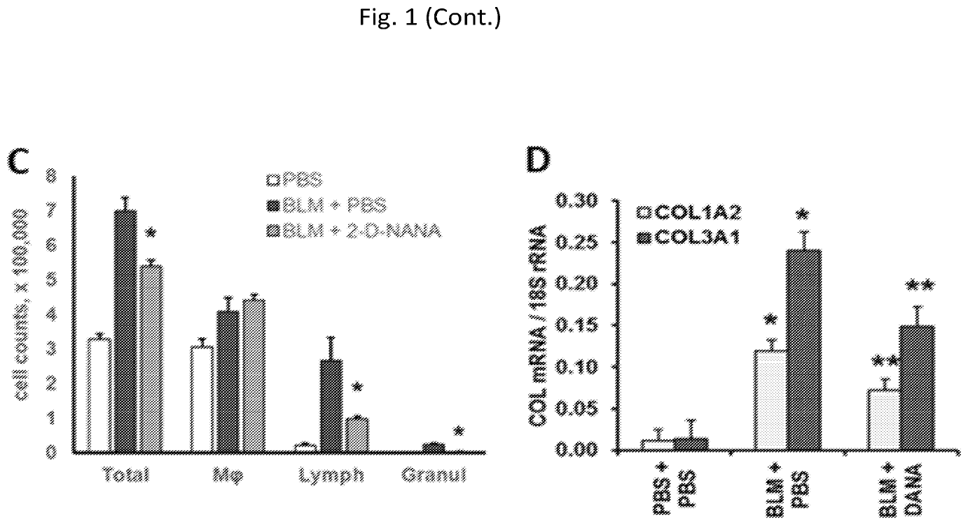

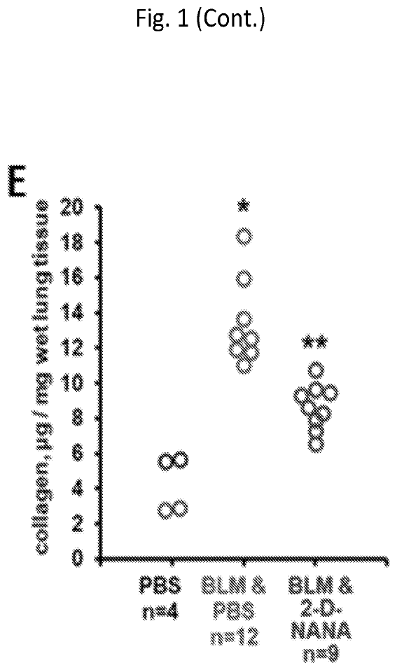

Mice were intraperitoneally injected with 15 mg/kg of 2DN dissolved in saline, or saline alone. After 24 h, the mice were re-injected with 2DN or the vehicle alone. At 18 h following the second injection, the mice were euthanized and the lungs were harvested, weighed, homogenized, centrifuged, and the supernatants processed for total protein levels and total NEU activity for the fluorogenic substrate, 4-MU-NANA (FIG. 1A). Administration of 2DN 24 h prior to harvesting lungs reduced total NEU activity by at least 8-fold. These data indicate the ability of a broad-spectrum sialidase inhibitor to diminish total lung NEU activity in vivo.

Effect of Broad-Spectrum Sialidase Inhibition on Lymphocyte Infiltration and Collagen Deposition in the Lungs of Bleomycin-Challenge Mice:

Mice challenged I.T. with bleomycin were serially weighed, and on days 8-14, daily administered i.p. 2DN or vehicle alone. On day 14, mice were sacrificed, and BALF and lungs harvested. The BALF was processed for total and differential cell counts, and lung tissues were collagenase-digested for lymphocyte counts and flow cytometry, processed for qRT-PCR for collagen mRNA expression, or homogenized for hydroxyproline-based assays to quantify collagen protein (FIG. 1C, 1D, 1E). In all bleomycin-challenged mice, total body weight decreased through days 1-7 (FIG. 1B). While the saline-injected, bleomycin-challenged mice continued to lose body weight through the 14-day study period, on days ≥9, the 2DN-treated, bleomycin-administered mice maintained and even increased body weight. By day 14, the mean±SE body weight for the 2DN-treated mice returned to their pretreatment baseline. On day 14, total and differential BALF cell counts were performed (FIG. 1C). In the PBS-injected, bleomycin-challenged mice, total cell counts, macrophages, lymphocytes, and neutrophils each were increased compared to BALF cell counts for the simultaneous saline controls. The addition of broad-spectrum NEU inhibition with 2DN reversed the increased in total cell counts, lymphocytes, and neutrophils, each compared to counts in the PBS-injected, bleomycin-challenged mice. In these same mice, lung homogenates were processed for qRT-PCR using primers for collagen 1A2 and collagen 3A1 to quantify their mRNA expression (FIG. 1D). The collagen transcripts were increased in lung tissues obtained from bleomycin-administered mice compared to those seen in lung tissues taken from the PBS controls. In those bleomycin-challenged mice that received 2DN, lung collagen mRNAs were reduced compared to those found in mice administered bleomycin alone. Finally, lung homogenates from these same mice were processed for hydroxyproline-based QuickZyme assays for collagen quantitation (FIG. 1E). As previously shown, bleomycin treatment dramatically increased collagen protein levels compared to those seen in the PBS controls. The 2DN treatment reversed the collagen levels in bleomycin-challenged mice compared to those seen in mice treated with bleomycin alone. These combined data indicate that broad-spectrum NEU inhibition with 2DN protects against total body weight loss, recruitment of inflammatory cells to the bronchoalveolar compartment, and increased collagen synthesis and deposition in the lung tissues of bleomycin-challenged mice. Multiple sialidases are known to be expressed In lung tissues. Which one or more of the 4 known mammalian sialidase(s) was operative in the profibrotic host response to bleomycin was unclear.

Sialidase and PPCA Expression in Lung Tissue from Bleomycin-Challenged Mice:

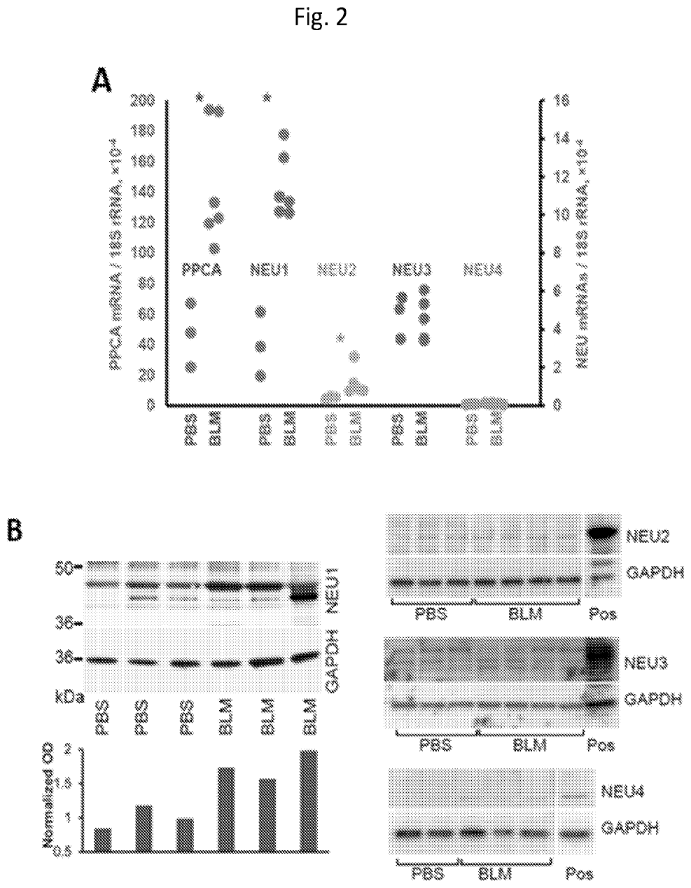

Since broad-spectrum sialidase inhibition protected against bleomycin-induced pulmonary fibrosis, we asked whether increased expression of one or more of the 4 known mammalian sialidase(s) might be involved. Mice were administered bleomycin or vehicle alone, and after 14 days, were sacrificed and lungs harvested and homogenized. The lung homogenates were processed for qRT-PCR with primers for NEU1, NEU2, NEU3, NEU4, and PPCA mRNAs (FIG. 2A) or quantitative immunoblotting probing for NEU and PPCA proteins (FIG. 2B). The lung tissues of bleomycin-challenged mice contained dramatically increased NEU1 and PPCA mRNA expression compared to that seen in the vehicle controls (FIG. 2A). No changes in NEU3 and -4 mRNA expression were detected. Similarly, bleomycin administration increased NEU1 protein expression in lung tissues compared to that seen in lung tissues from the vehicle controls (FIG. 2B). NEU2, -3, and -4 protein expression remained unchanged. These combined data implicate NEU1, together with its chaperone, PPCA, as the operative sialidase in the pulmonary response to bleomycin. However, the participation of one or more of the other three known mammalian sialidases cannot be absolutely excluded.

NEU1-Selective Inhibition of Sialidase Activity in Human Lung Cells In Vitro:

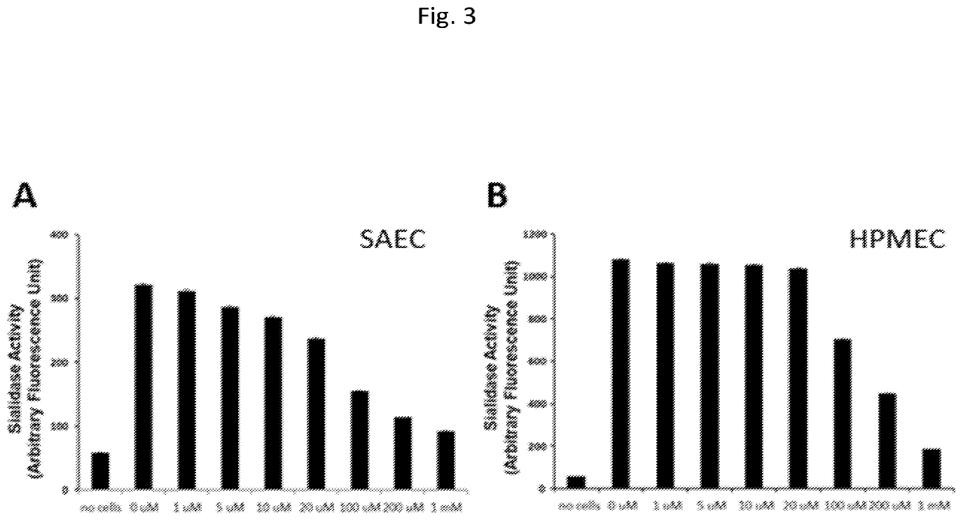

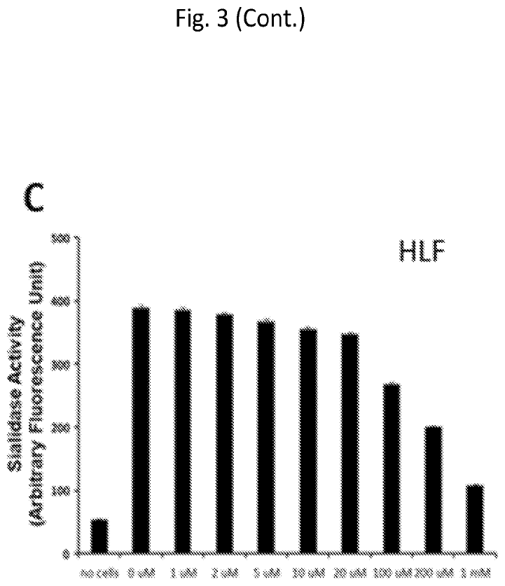

We previously reported 2DN- and C9-BADANA-mediated inhibition of sialidase activity in human airway epithelia, lung microvascular endothelia, and lung fibroblasts. Here, fixed numbers of SAECs (FIG. 3A), HPMECs (FIG. 3B), or HLFs (FIG. 3C) were assayed for sialidase activity for the 4-MU-NANA substrate in the presence of increasing concentrations of the NEU1-selective inhibitor III-32B5. In all three lung cell types III-32B5 dose-dependently inhibited sialidase activity (FIGS. 3A-C). The NEU1 inhibitor can comprise C1-methyl esters of the compounds.

Effect of NEU1-Selective Pharmacologic Inhibition on Total Sialidase Activity in Mouse Lung In Vivo:

Mice were injected ip with 15 mg/kg of C9-BADANA, or III-32B5, or saline alone. After 24 h, the mice were administered these same injections, and 18 h later, were euthanized and their BALFs and lungs harvested. The lungs were processed for total protein levels and total sialidase activity. Pretreatment with C9-BADANA and III-32B5 reduced total lung sialidase activity. The BALFs from these same mice were processed for cell counts and sialidase activity. Pretreatment with C9-BADANA and III-32B5 reduced total BALF cell sialidase activity. These findings establish the ability of each of 2 NEU1-selective pharmacologic inhibitors to profoundly reduce total lung and BALF cell sialidase activity in vivo. The NEU1 inhibitor can comprise C1-methyl esters of the compounds.

Effect of NEU1-Selective Sialidase Inhibition on Lymphocyte Infiltration and Collagen Deposition in the Lungs of Bleomycin-Challenged Mice:

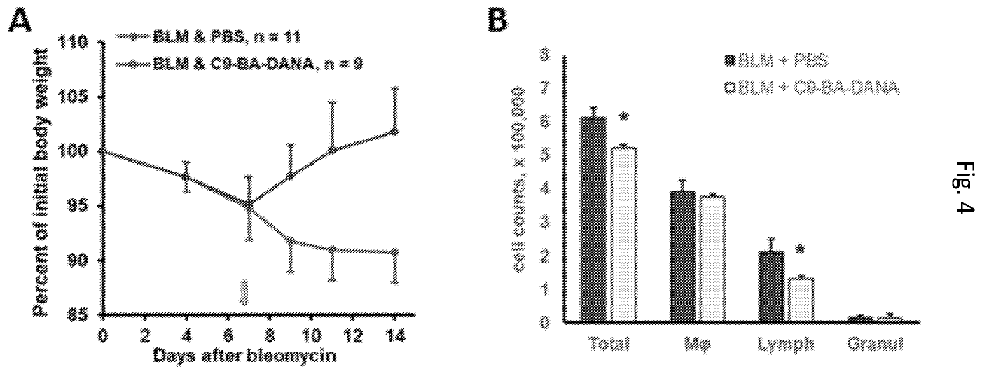

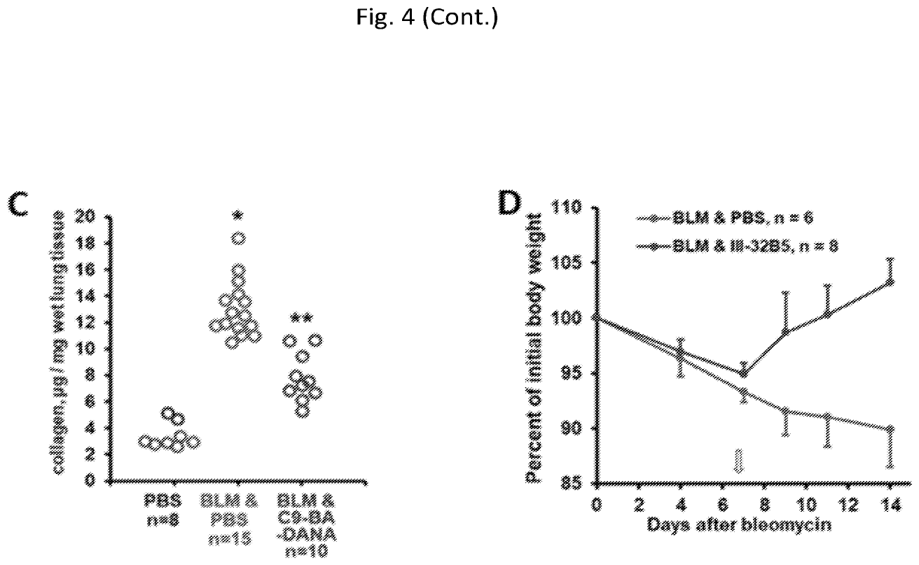

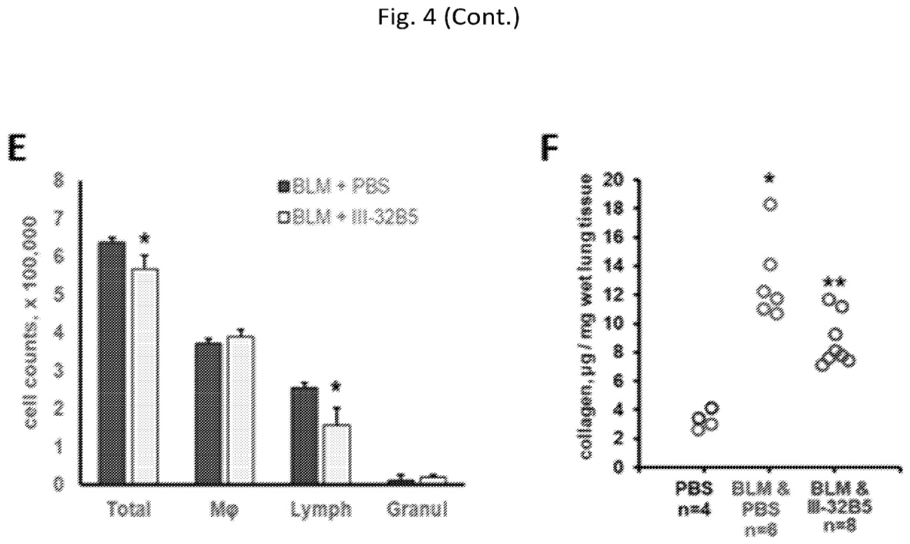

The bleomycin challenge selectively increased NEU1 expression (FIG. 2A-B). With this in mind, mice challenged with bleomycin were serially weighed, and on days 8-14, daily administered IP, either of 2 NEU1-selective NEU inhibitors, C9-BA-DANA or III-32B5, or vehicle alone. On day 14, mice were sacrificed and BALF and lungs harvested. The BALF was processed for total and differential cell counts and lung tissues collagen-digested for lymphocyte counts and flow cytometry, or homogenized and processed for qRT-PCR for collagen mRNA expression or hydroxyproline-based assays to quantify collagen protein (FIG. 4B, 4C, 4E, 4F). As shown above (FIG. 1B), in bleomycin-challenged, vehicle-administered mice, total body weight decreased through the 14-day study period (FIGS. 4A and 4D). On days ≥9, those mice that received either C9-BADANA or III-32B5, maintained and even increased their body weight. By day 14, these same 2 groups of mice increased their mean body weights, returning to their pretreatment baselines. Administration of each of the 2 NEU1-selective sialidase inhibitors reversed the bleomycin-provoked increased in BALF total cell counts, lymphocytes, and neutrophils (FIGS. 4B and 4E). Each of the inhibitors protected against the bleomycin-induced increases in collagen mRNA and protein (FIGS. 4C and 4F) expression. Taken together, these data indicate that NEU1-selective sialidase inhibition protects against the increased lymphocyte infiltration and collagen deposition within the lungs of bleomycin-challenged mice. The substrates through which NEU1 catalytic activity might regulate the intrapulmonary profibrotic response were unclear. The NEU1 inhibitor can comprise C1-methyl esters of the compounds.

### CONCLUSIONS

Mice challenged with bleomycin exhibit (a) decreased total body weight, (b) increased macrophages, lymphocytes, and neutrophils in the bronchoalveolar compartment, and (c) increased collagen expression in lung tissues.

Broad spectrum sialidase inhibition with 2DN reverses weight loss, T-lymphocyte recruitment, and collagen deposition in bleomycin-challenged mice.

Bleomycin administration increases lung tissue expression of NEU1, but not NEU2, -3, or -4.

NEU1-selective sialidase inhibition protects against bleomycin-provoked weight loss, T-lymphocyte recruitment, and collagen deposition.

### Example 2. NEU1-Selective Sialidase Inhibitors for Treatment of Idiopathic Pulmonary Fibrosis

Idiopathic Pulmonary Fibrosis (IPF) is an unrelenting, uniformly fatal disease in which lung architecture is distorted and replaced by scar tissue, thereby compromising pulmonary function. IPF patients only survive 2 to 3 years after diagnosis. Although numerous host factors have been implicated in IPF pathogenesis, no unified mechanistic model or life-saving therapy has been established. Recently, we demonstrated elevated NEU1 sialidase expression in the lung tissues of IPF patients, including in the airway epithelium, vascular endothelium, and lung fibroblasts. We found that forced NEU1 overexpression restrained cell migration of and wound healing by primary human small airway epithelial cells, disrupted in vitro angiogenesis by lung microvascular endothelia, and increased collagen expression by lung fibroblasts. Each of these NEU1-driven phenotypes is consistent with IPF pathophysiology. We then found that gene delivery of NEU1 to mouse lungs in vivo increased collagen deposition comparable to that observed in the classical bleomycin (BLM) model of lung fibrosis. In this example, we build on the observation that NEU1-selective sialidase inhibitors can dramatically protect against BLM-provoked pulmonary fibrosis. We propose preclinical development of one of our lead drug candidates, C9-BADANA, as a therapeutic intervention for IPF. This Example addresses 4 milestones: 1) In mice, establish the dose-response relationship between C9-BADANA dosing and lung sialidase inhibition, 2) Define the duration of sialidase inhibitory activity in lung tissues, 3) Determine the feasibility of orally dosed C9-BADANA, and finally, 4) Establish a dosing regimen for C9-BADANA that protects against BLM-provoked pulmonary fibrosis.

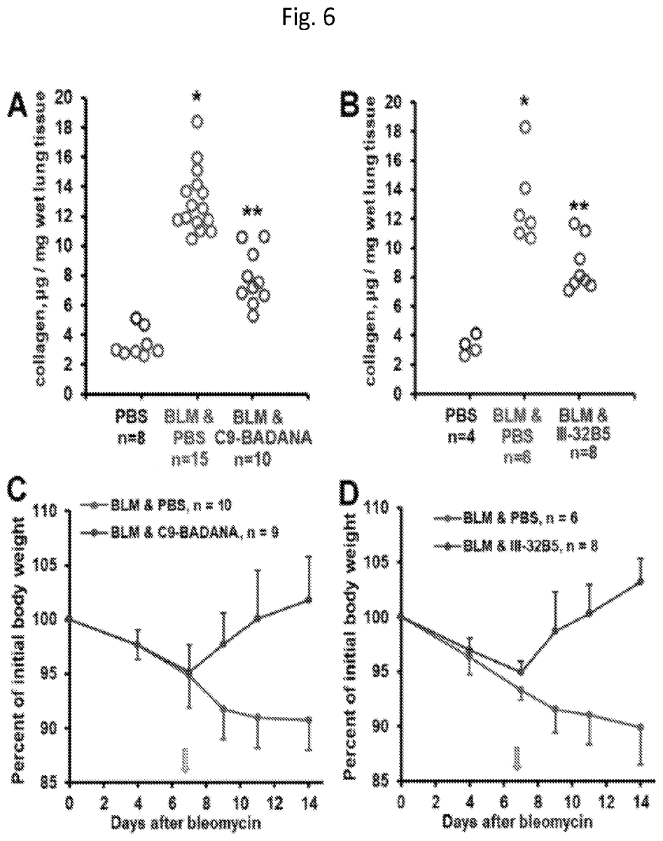

This technology is a small-molecule treatment for an invariably fatal disease, Idiopathic Pulmonary Fibrosis (IPF). We discovered that expression of the sialidase, neuraminidase-1 (NEU1), is elevated in the lungs of IPF patients (Luzina et al., Am J Physiol Lung Cell Mol Physiol 310:L940-L954, 2016.). Further, forced NEU1 overexpression in the lungs of mice recapitulates the intra-pulmonary lymphocyte infiltration and collagen deposition that are hallmarks of IPF pathophysiology (Luzina et al., Am J Physiol Lung Cell Mol Physiol 310:L940-L954, 2016.). Our collaborators in Japan and Canada have created the only two known NEU1-selective inhibitors, C9-BADANA (Magesh et al., Bioorg Med Chem Lett, 18:532-537, 2008.; Hyun et al., Glycobiology 26:834-849, 2016.) and III-32B5, respectively (Guo et al., J Med Chem 61:11261-11279, 2018.). Lead candidate C9-BADANA (FIG. 5) represents a promising approach to stopping—even reversing—the course of IPF (Magesh et al., Bioorg Med Chem Lett, 18:532-537, 2008.; Hyun et al., Glycobiology 26:834-849, 2016.) (FIGS. 6A-B). The NEU1 inhibitor can comprise C1-methyl esters of the compounds.

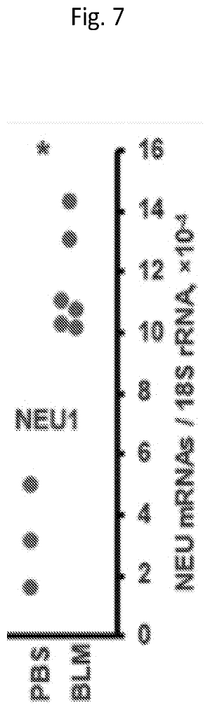

Results: NEU1 is one of four mammalian neuraminidases, each with overlapping tissue distributions, substrates, and functions. To protect normal physiology, the sole isozyme implicated in IPF must be targeted. The IC₅₀ of C9-BADANA is 10 μM for NEU1, but >1000 μM for NEUs 2-4 (Magesh et al., Bioorg Med Chem Lett, 18:532-537, 2008.). This NEU1-inhibitory activity holds for primary human lung cells, and for murine lungs, in vivo (Hyun et al., Glycobiology 26:834-849, 2016.). Compound III-32B5 (C5-hexanamido-C9-acetamido-DANA) has a similar profile (Guo et al., J Med Chem 61:11261-11279, 2018.) and can be evaluated as described for C9-BADANA below. Elevated expression of NEU1 in fibrotic lungs points to this isozyme as a potentially druggable target (FIG. 7). In fact, we have shown that NEU1-selective inhibitors dramatically reduce intra-pulmonary collagen deposition in bleomycin (BLM)-challenged mice (FIG. 6A-B). Of note, none of the other three members of the NEU family were implicated in the process of lung fibrosis.

Milestone 1. Establish the dose-response relationship between C9-BADANA and inhibition of lung sialidase activity. Our data indicate that C9-BADANA at 15 μg/kg body weight dramatically reduces lung sialidase activity and collagen deposition (FIG. 6A)(Hyun et al., Glycobiology 26:834-849, 2016.). Female C57BL/6 mice (10-12 weeks) weighing 18-20 g, are treated in accordance with our IACUC-approved protocol (#1218015). Seven groups of mice are given a single intraperitoneal injection of C9-BADANA, with doses 0 (vehicle control), 1, 5, 10, 15, 25, and 50 μg/kg BW. After 24 hours, mice are euthanized by CO₂ asphyxiation and cervical dislocation, as we described (Pochetuhen et al., Am J Pathol 171:428-437, 2007.; Luzina et al., J Pharmacol Exp Ther 355:13-22, 2015.; Wyman et al., Am J Physiol Lung Cell Mol Physiol 312:L945-L958, 2017.). Lungs are immediately harvested and processed for measurement of sialidase activity for the fluorogenic substrate 2′-(4-methylumbelliferyl)-α-D-N-acetylneuraminic acid (4-MU-NANA), as we described (Hyun et al., Glycobiology 26:834-849, 2016.; Lillehoj et al., J Biol Chem 287:8214-8231, 2012.; Lee et al., J Biol Chem 289:9121-9135, 2014.; Lillehoj et al., J Biol Chem 290:18316-18331, 2015.; Lillehoj et al., J Biol Chem 294:662-678, 2019.).

For these studies and the following milestones, a preliminary power analysis was performed by Dr. Soren Bentzen of the UMB Biostatistics Core. It was based on the 14-day collagen deposition data in the bleomycin (BLM) model, where BLM-challenged mice and mice receiving both BLM and C9-BADANA showed mean values of 13.4±2.1 μg/mg and 8.6±1.7 μg/mg, respectively—a large effect size (FIG. 6A). Setting significance α=0.05 and using a two-sided unequal-variance t-test, a sample size of 5 achieved 92% power. Thus, 5 mice per group can be used in all animal experiments. This milestone can establish the minimal effective dose, the maximal saturating dose, and allow the IC₅₀ to be calculated.

Milestone 2. Estimate the in vivo half-life of C9-BADANA by defining the duration of sialidase inhibitory activity in lungs after I.P. administration of a single dose. Mice are injected with one of four doses, determined by Milestone 1: vehicle, the minimal effective dose, IC₅₀, and maximally effective dose. After 2, 6, 18, 24, and 48 hours, mice are euthanized and lungs processed for total sialidase activity. For each timepoint after each dose, 5 mice can be studied. For this initial estimate of t_{1/2}, we are focusing on the effects of the drug on the critical organ—the lung—rather than on quantitating the systemic concentration of the drug. An unexpectedly short half-life (<˜6 hours) would suggest that twice-daily dosing may be required (100 mice).

Milestone 3. Determine the feasibility of orally-dosed C9-BADANA. Based on the intraperitoneal dose established in Milestone 1 and estimating oral availability to be in the range of 10% to 100%, three experimental groups can be used: 15 μg/kg (100%), 45 μg/kg (30%), and 150 μg/kg (10%). C9-BADANA is given by gastric lavage. At t=0 and after 2, 6, 18, 24, and 48 hours, mice are euthanized and lungs processed for total sialidase activity. For the positive control, i.p.-administered C9-BADANA groups from Milestone 2 are used. If oral availability is <10%, subcutaneous or intravenous dosing can be used instead.

Milestone 4. Establish the dosing regimen that protects mice against BLM-induced lung fibrosis. At day 0, a single dose of 0.075 U of BLM is delivered intra-tracheally to C57BL/6 mice via a MicroSprayer, as we described (Nalysynk et al., Eur Respir Rev 126:355-361, 2012.; DiMasi et al., Journal of Health Economics 47:20-33, 2016.; Pochetuhen et al., Am J Pathol 171:428-437, 2007.). On day 8 post-BLM, a dosing regimen for C9-BADANA based on the results of Milestones 1-3 is initiated, with vehicle as a control group. Key decisions will include: frequency (e.g. 1× or 2× per day; every other day); effective dose (e.g. 15 μg/kg), and route of administration (e.g. oral vs. i.p.). On day 14, mice are euthanized, and total collagen lung content measured with hydroxyproline-based QuickZyme assay (Luzina et al., Am J Physiol Lung Cell Mol Physiol 310:L940-L954, 2016.; Pochetuhen et al., Am J Pathol 171:428-437, 2007.; Luzina et al., J Pharmacol Exp Ther 355:13-22, 2015.; Wyman et al., Am J Physiol Lung Cell Mol Physiol 312:L945-L958, 2017.). Although more expensive than assaying sialidase, the measure of collagen is the Gold Standard for evaluating lung fibrosis. Two (or more) independent experiments can be performed. (40 mice).

1. A method for treating a fibrotic lung disease or fibrotic lung condition in a subject that involves an increase in NEU1 expression and/or activity, comprising administering to the subject an effective amount of an agent that inhibits the activity of NEU1 sialidase, thereby treating the fibrotic lung disease or fibrotic lung condition in the subject.

2. The method of claim 1, wherein the agent is a NEU1-selective inhibitor.

3. The method of claim 1, wherein the fibrotic lung disease or fibrotic lung condition is idiopathic pulmonary fibrosis.

4. The method of claim 1, wherein the fibrotic lung disease or fibrotic lung condition is associated with a connective tissue disorder.

5. The method of claim 1, wherein the fibrotic lung disease or fibrotic lung condition is selected from the group consisting of sarcoidosis, allergic pneumonia, pneumoconiosis, drug-induced fibrosis, radiation-induced fibrosis, noxious chemical compound-induced fibrosis, and fibrogenic alveolitis associated with collagen vascular disease.

6. The method of claim 1, wherein the agent reduces or prevents myofibroblast accumulation in the subject.

7. The method of any of claim 1, wherein the agent reduces dyspnea caused by the fibrotic lung disease or condition in the subject.

8. The method of claim 2, wherein the NEU1-selective inhibitor is compound C9-BADANA.

9. The method of claim 2, wherein the NEU1-selective inhibitor is compound III-32B5.

10. (canceled)

11. The method of claim 1 wherein the agent comprises a nucleic acid molecule comprising a sequence that binds to at least a portion of a nucleotide sequence of NEU1.

12. The method of claim 11, wherein the nucleotide sequence of NEU1 is SEQ ID NO: 1.

13-15. (canceled)

16. The method of claim 1, wherein the agent comprises a small interfering RNA (siRNA) molecule.

17. The method of claim 1, wherein the agent comprises a small hairpin RNA (shRNA) molecule.

18-19. (canceled)

20. The method of claim 1, wherein the agent comprises an expression vector, wherein the vector is a viral vector or a non-viral vector.

21. The method of claim 20, wherein the viral vector is an adenoviral vector, an adeno-associated viral vector, a lentiviral vector, or a retroviral vector.

22. (canceled)

23. A composition for treating a fibrotic lung disease or fibrotic lung condition in a subject, the composition comprising an effective amount of an agent that inhibits the activity of NEU1 sialidase and a pharmaceutically acceptable carrier.

24. The composition of claim 23, wherein the composition comprises a nucleic acid molecule that comprises a nucleotide sequence that binds to at least a portion of a nucleotide sequence of NEU1.

25-28. (canceled)

29. The composition of claim 23, wherein the composition comprises a small interfering RNA (siRNA) molecule.

30. The composition of claim 23, wherein the composition comprises a small hairpin RNA (shRNA) molecule.

31. (canceled)

32. The composition of claim 23, wherein the composition comprises an expression vector, wherein the vector is a viral vector or a non-viral vector.

33-34. (canceled)


SEQUENCE LISTING (2 sequences)

SEQ ID NO: 1 - DNA, 1248 bp, Homo sapiens
atgactggggagcgacccagcacggcgctcccggacagacgctgggggccgcggattctgggcttctggggaggctgtagggtttgggtgtttgccgcgatcttcctgctgctgtctctggcagcctcctggtccaaggctgagaacgacttcggtctggtgcagccgctggtgaccatggagcaactgctgtgggtgagcgggagacagatcggctcagtggacaccttccgcatcccgctcatcacagccactccgcggggcactcttctcgcctttgctgaggcgaggaaaatgtcctcatccgatgagggggccaagttcatcgccctgcggaggtccatggaccagggcagcacatggtctcctacagcgttcattgtcaatgatggggatgtccccgatgggctgaaccttggggcagtagtgagcgatgttgagacaggagtagtatttcttttctactccctttgtgctcacagggccggctgccaggtggcctctaccatgttggtatggagcaaggatgatggtgtttcctggagcacaccccggaatctctccctggatattggcactgaagtgtttgcccctggaccgggctctggtattcagaaacagcgggagccacggaagggccgcctcatcgtgtgtggccatgggacgctggagcgggacggagccttctgtctcctcagcgatgatcatggtgcctcctggcgctacggaagtggggtcagcggcatcccctacggtcagcccaagcaggaaaatgatttcaatcctgatgaatgccagccctatgagctcccagatggctcagtcgtcatcaatgcccgaaaccagaacaactaccactgccactgccgaattgtcctccgcagctatgatgcctgtgatacactaaggccccgtgatgtgaccttcgaccctgagctcgtggaccctgtggtacctgcaggagctgtagtcaccagctccggcattgtcttcttctccaacccagcacatccagagttccgagtgaacctgaccctgcgatggagcttcagcaatggtacctcatggcggaaagagacagtccagctatggccaggccccagtggctattcatccctggcaaccctggagggcagcatggatggagaggagcaggccccccagctctacgtcctgtatgagaaaggccggaaccactacacagagagtatctccgtggccaaaatcagtgtctatgggacactttaa

SEQ ID NO: 2 - protein, 415 aa, Homo sapiens [sequence omitted]